(19) United States
(12) Patent Application Publication
(10) Pub. No.: US 2014/0363864 A1
(43) Pub. Date: Dec. 11, 2014

(54) MICROORGANISMS AND METHODS FOR THE PRODUCTION OF 1,4-CYCLOHEXANEDIMETHANOL

(71) Applicant: Genomatica, Inc., San Diego, CA (US)

(72) Inventors: Priti Pharkya, San Diego, CA (US); Mark J. Burk, San Diego, CA (US)

(73) Assignee: Genomatica, Inc., San Diego, CA (US)

(21) Appl. No.: 14/152,799

(22) Filed: Jan. 10, 2014

Related U.S. Application Data
(63) Continuation of application No. 13/327,385, filed on Dec. 15, 2011, now abandoned.
(60) Provisional application No. 61/424,592, filed on Dec. 17, 2010.

(51) Int. Cl. C12P 7/02; C12N 15/81; C12N 15/78; C12N 15/75; C12N 15/76; C12N 15/74; C12N 15/70

(52) U.S. Cl. CPC C12P 7/02; C12N 15/74; C12N 15/81; C12N 15/70; C12N 15/75; C12N 15/76; C12N 15/78; C12N 15/815
USPC 435/155; 435/252.3; 435/254.2; 435/254.11; 435/252.33; 435/252.2; 435/252.31; 435/252.35; 435/252.34; 435/254.21; 435/254.3; 435/254.23; 435/252.32; 435/254.22

(57) ABSTRACT

A non-naturally occurring microbial organism includes a microbial organism having a 1,4-cyclohexanedimethanol pathway that includes at least one exogenous nucleic acid encoding a 1,4-cyclohexanedimethanol pathway enzyme expressed in a sufficient amount to produce 1,4-cyclohexanedimethanol. A method for producing 1,4-cyclohexanedimethanol includes culturing a non-naturally occurring microbial organism having a 1,4-cyclohexanedimethanol pathway. The pathway includes at least one exogenous nucleic acid encoding a 1,4-cyclohexanedimethanol pathway enzyme expressed in a sufficient amount to produce 1,4-cyclohexanedimethanol, under conditions and for a sufficient period of time to produce 1,4-cyclohexanedimethanol.

This application is a continuation of U.S. Ser. No. 13/327,385 filed Dec. 15, 2011, which claims the benefit of priority of U.S. Ser. No. 61/424,592, filed Dec. 17, 2010, the entire contents of which are incorporated herein by reference.

### BACKGROUND OF THE INVENTION

This invention relates generally to the production of commodity and specialty chemicals and, more specifically to an integrated bioprocess for producing 1,4-cyclohexanedimethanol.

1,4-cyclohexanedimethanol (CHDM) is a glycol used for the production of polyesters. Polyester polymers derived from a diol composition utilizing 1,4-cyclohexanedimethanol have low melt viscosities and can be easily processed at acceptable glass transition temperatures for the preparation of powder coatings with a useful balance of chemical resistance, hardness and flexibility together with excellent appearance.

Currently CHDM is produced from dimethyl terephthalate (DMT) via a two-step hydrogenation reaction. The first hydrogenation reaction is ring saturation that produces dimethyl 1,4-cyclohexane dicarboxylate from DMT. The second one is reduction of the ester to produce CHDM. There exists a need for alternate routes for the preparation of 1,4-cyclohexanedimethanol. The present invention satisfies this need and provides related advantages as well.

The proposed pathways for the production of CHDM proceed via p-toluate which in turn can be produced from G3P and PEP. If p-toluate is further converted to dihydroxymethyl benzene (DHMB) by the pathways described in FIG. 1, the predicted yield from glucose is 0.57 mol/mol (0.44 g/g) assuming no carbon is used for energy production. In this case, the transformation of p-toluate to dihydroxymethyl benzene requires three reducing equivalents and one or two ATP molecules according to the net reaction:

p-toluate+O₂+(1 or 2)ATP+3NAD(P)H+3H⁺→DHMB+(1 or 2)ADP+(1 or 2)Pi+3NAD(P)⁺

Assuming a net consumption of 2 ATP molecules for producing 1 mole of DHMB, the maximum theoretical yield of DHMB is 0.52 mol/mol (0.4 g/g) glucose consumed.

If p-toluate is converted directly to CHDM by the pathway described in FIG. 2, the predicted yield from glucose is 0.5 mol/mol (0.4 g/g). If carbon is used to generate ATP, the yield drops down to 0.48 mol/mol glucose (0.38 g/g). In this case, the transformation of p-toluate to CHDM requires six reducing equivalents and one ATP molecule according to the net reaction:

p-toluate+O₂+ATP+6NAD(P)H+6H⁺→CHDM+ADP+Pi+6NAD(P)⁺+H₂O

### SUMMARY OF THE INVENTION

In some aspects, embodiments disclosed herein relate to a non-naturally occurring microbial organism that includes a microbial organism having a 1,4-cyclohexanedimethanol pathway having at least one exogenous nucleic acid encoding a 1,4-cyclohexanedimethanol pathway enzyme expressed in a sufficient amount to produce 1,4-cyclohexanedimethanol. The 1,4-cyclohexanedimethanol pathway includes an enzyme set selected from (1) a p-toluate monooxygenase, a p-hydroxymethyl benzoate reductase, a dihydroxymethyl benzene dehydrogenase; (2) a p-toluate kinase, a (p-methylbenzoyloxy)phosphonate reductase (dephosphorylating), a p-methylbenzyl alcohol dehydrogenase, and a p-methylbenzyl alcohol monooxygenase; (3) a p-methylbenzoyl-CoA synthetase, transferase and/or hydrolase, a phosphotrans-p-methylbenzoylase, a (p-methylbenzoyloxy)phosphonate reductase (dephosphorylating), a p-methylbenzyl alcohol dehydrogenase, and a p-methylbenzyl alcohol monooxygenase; (4) a p-methylbenzoyl-CoA synthetase, transferase and/or hydrolase, a p-methylbenzoyl-CoA reductase, a p-methylbenzyl alcohol dehydrogenase, and a p-methylbenzyl alcohol monooxygenase; (5) a p-toluate reductase, a p-methylbenzyl alcohol dehydrogenase, and a p-methylbenzyl alcohol monooxygenase; (6) a p-toluate monooxygenase, a p-hydroxymethyl benzoyl-CoA synthetase, transferase and/or hydrolase, a p-hydroxymethyl benzoyl-CoA reductase, a dihydroxymethyl benzene dehydrogenase, and chemical reduction; (7) a p-toluate monooxygenase, a p-hydroxymethyl benzoate kinase, a (p-hydroxymethylbenzoyloxy)phosphonate reductase (dephosphorylating), a dihydroxymethyl benzene dehydrogenase, and chemical reduction; (8) a p-toluate monooxygenase, a p-hydroxymethyl benzoate kinase, a phosphotrans-p-hydroxymethylbenzoylase, a p-hydroxymethyl benzoyl-CoA reductase, a dihydroxymethyl benzene dehydrogenase, and chemical reduction; (9) a p-toluate monooxygenase, a p-hydroxymethyl benzoate kinase, a phosphotrans-p-hydroxymethylbenzoylase, a p-hydroxymethyl benzoyl-CoA reductase, a 4-hydroxymethyl cyclohex-1,5-diene carboxyl CoA reductase, a 4-hydroxymethyl cyclohex-1-ene carboxyl-CoA reductase, a 4-hydroxymethylcyclohexanoyl-CoA reductase, and a 4-hydroxymethyl cyclohexane carbaldehyde reductase; and (10) a p-toluate monooxygenase, a p-hydroxymethyl benzoyl-CoA synthetase, transferase and/or hydrolase, a p-hydroxymethyl benzoyl-CoA reductase, a 4-hydroxymethyl cyclohex-1,5-diene carboxyl CoA reductase, a 4-hydroxymethyl cyclohex-1-ene carboxyl-CoA reductase, a 4-hydroxymethylcyclohexanoyl-CoA reductase, and a 4-hydroxymethyl cyclohexane carbaldehyde reductase.

In some aspects, embodiments disclosed herein relate to a method for producing 1,4-cyclohexanedimethanol that includes culturing a non-naturally occurring microbial organism having a 1,4-cyclohexanedimethanol pathway. The pathway includes at least one exogenous nucleic acid encoding a 1,4-cyclohexanedimethanol pathway enzyme expressed in a sufficient amount to produce 1,4-cyclohexanedimethanol, under conditions and for a sufficient period of time to produce 1,4-cyclohexanedimethanol. The 1,4-cyclohexanedimethanol pathway includes an enzyme set selected from (1) a p-toluate monooxygenase, and a p-hydroxymethyl benzoate reductase, a dihydroxymethyl benzene dehydrogenase; (2) a p-toluate kinase, a (p-methylbenzoyloxy)phosphonate reductase (dephosphorylating), a p-methylbenzyl alcohol dehydrogenase, and a p-methylbenzyl alcohol monooxygenase; (3) a p-methylbenzoyl-CoA synthetase, transferase and/or hydrolase, a phosphotrans-p-methylbenzoylase, a (p-methylbenzoyloxy)phosphonate reductase (dephosphorylating), a p-methylbenzyl alcohol dehydrogenase, and a p-methylbenzyl alcohol monooxygenase; (4) a p-methylbenzoyl-CoA synthetase, transferase and/or hydrolase, a p-methylbenzoyl-CoA reductase, a p-methylbenzyl alcohol dehydrogenase, and a p-methylbenzyl alcohol monooxygenase; (5) a p-toluate reductase, a p-methylbenzyl alcohol dehydrogenase, and a p-methylbenzyl alcohol monooxygenase; (6) a p-toluate monooxygenase, a p-hydroxymethyl benzoyl-CoA synthetase, transferase and/or hydrolase, a p-hydroxymethyl benzoyl-CoA reductase, a dihydroxymethyl benzene dehydrogenase, and chemical reduction; (7) a p-toluate monooxygenase, a p-hydroxymethyl benzoate kinase, a (p-hydroxymethylbenzoyloxy)phosphonate reductase (dephosphorylating), a dihydroxymethyl benzene dehdyrogenase, and chemical reduction; (8) a p-toluate monooxygenase, a p-hydroxymethyl benzoate kinase, a phosphotrans-p-hydroxymethylbenzoylase, a p-hydroxymethyl benzoyl-CoA reductase, a dihydroxymethyl benzene dehdyrogenase, and chemical reduction; (9) a p-toluate monooxygenase, a p-hydroxymethyl benzoate kinase, a phosphotrans-p-hydroxymethylbenzoylase, a p-hydroxymethyl benzoyl-CoA reductase, a 4-hydroxymethyl cyclohex-1,5-diene carboxyl CoA reductase, a 4-hydroxymethyl cyclohex-1-ene carboxyl-CoA reductase, a 4-hydroxymethylcyclohexanoyl-CoA reductase, and a 4-hydroxymethyl cyclohexane carbaldehyde reductase; and (10) a p-toluate monooxygenase, a p-hydroxymethyl benzoyl-CoA synthetase, transferase and/or hydrolase, a p-hydroxymethyl benzoyl-CoA reductase, a 4-hydroxymethyl cyclohex-1,5-diene carboxyl CoA reductase, a 4-hydroxymethyl cyclohex-1-ene carboxyl-CoA reductase, a 4-hydroxymethylcyclohexanoyl-CoA reductase, and a 4-hydroxymethyl cyclohexane carbaldehyde reductase.

### BRIEF DESCRIPTION OF THE DRAWINGS

FIG. 1 shows the pathways for the conversion of p-toluate to dihydroxymethyl benzene. The enzymes are: A. p-toluate monooxygenase, B. p-hydroxymethyl benzoate reductase, C. dihydroxymethyl benzene dehydrogenase, D. p-methylbenzoyl-CoA synthetase, transferase and/or hydrolase, E. p-toluate kinase, F. phosphotrans-p-methylbenzoylase, G. (p-methylbenzoyloxy)phosphonate reductase (dephosphorylating), H. p-methylbenzyl alcohol dehydrogenase, I. p-methylbenzoyl-CoA reductase, J. p-toluate reductase, and K. p-methylbenzyl alcohol monooxygenase, L. p-hydroxymethyl benzoyl-CoA synthetase, transferase and/or hydrolase, M. p-hydroxymethyl benzoyl-CoA reductase. N. p-hydroxymethyl benzoate kinase. O. phosphotrans-p-hydroxymethylbenzoylase, and P. (p-hydroxymethyl-benzoyloxy)phosphonate reductase (dephosphorylating).

FIG. 2 shows the pathways for the conversion of p-hydroxymethyl benzoyl-CoA to 1,4-cyclohexanedimethanol. The enzymes are: A. p-hydroxymethyl benzoyl-CoA reductase, B. 4-hydroxymethyl cyclohex-1,5-diene carboxyl CoA reductase, C. 4-hydroxymethyl cyclohex-1-ene carboxyl-CoA reductase, D. 4-hydroxymethylcyclohexanoyl-CoA reductase, and E. 4-hydroxymethyl cyclohexane carbaldehyde reductase.

### DETAILED DESCRIPTION OF THE INVENTION

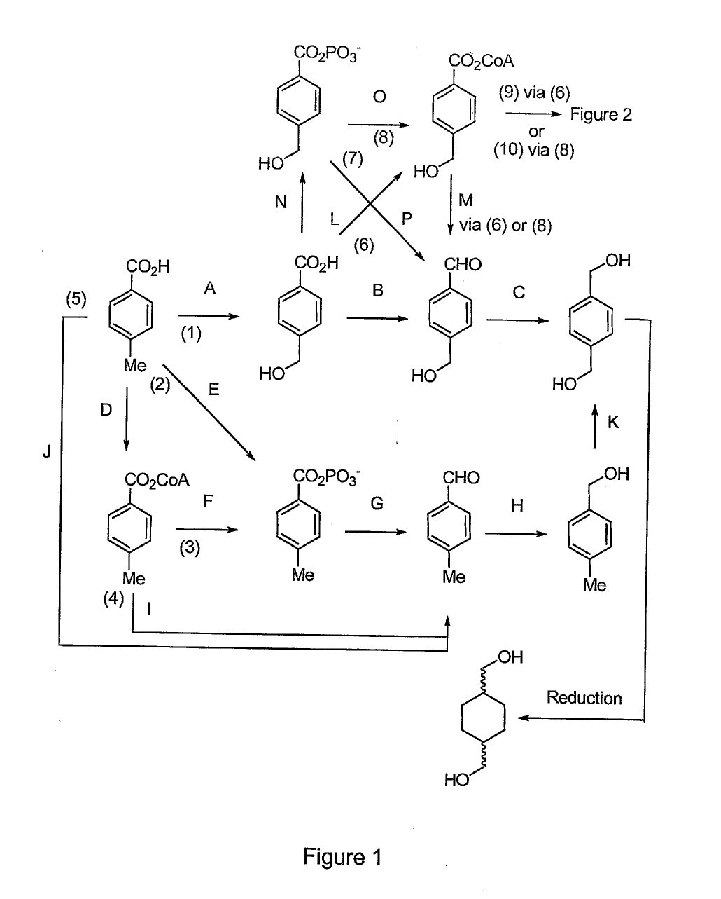

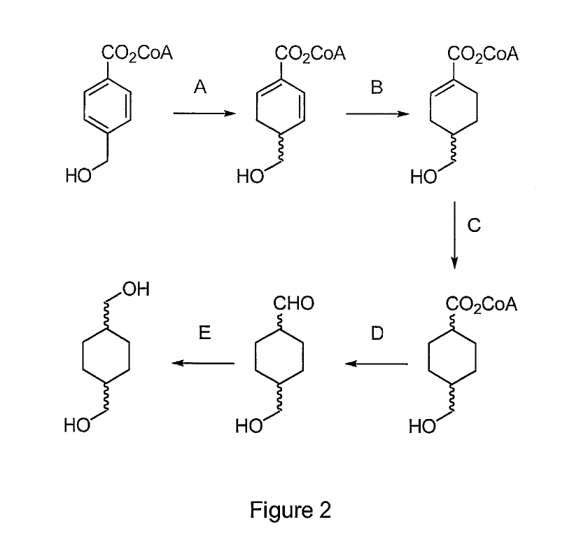

The present invention is directed to the design and production of cells and organisms having biosynthetic production capabilities for 1,4-cyclohexanedimethanol. Biosynthetic production of 1,4-cyclohexanedimethanol can be confirmed by construction of strains having the designed metabolic genotype. Pathways for the production of 1,4-cyclohexanedimethanol (CHDM) can utilize renewable feedstocks such as sugars and syngas. Intermediate p-toluate can be produced from sugars using central metabolic precursors, pyruvate and glyceraldehydes-3-phosphate. As disclosed herein, a number of metabolic pathways for the production of 1,4-cyclohexanedimethanol are described with reference to the pathways shown in FIG. 1 and FIG. 2. One such route proceeds via initial oxidation of the side chain methyl group of p-toluate and subsequent reduction of the carboxylic acid groups. Other disclosed routes proceed via transformations that first convert the carboxylic acid functionality of p-toluate to an alcohol, and then rely on late stage oxidation of the side chain methyl group of p-methylbenzyl alcohol. The final step, the reduction of aromatic system in dihydroxymethyl benzene, can be achieved either enzymatically or via chemical reduction. In yet further alternative pathways, organisms designed for the production of p-toluate can be used to access CHDM via reduction of the aromatic system prior to reduction of the carboxy group. An exemplary route of this type is shown in FIG. 2 which proceeds via stepwise reduction of the aromatic system from p-hydroxymethlybenzoyl-CoA. Reduction to the product CHDM can occur with or without control of the two newly created stereogenic centers. Methods for enhancing the ratio of CHDM isomers are known in the art.

In some embodiments, the present invention provides a non-naturally occurring microbial organism having a 1,4-cyclohexanedimethanol pathway that includes at least one exogenous nucleic acid encoding a 1,4-cyclohexanedimethanol pathway enzyme expressed in a sufficient amount to produce 1,4-cyclohexanedimethanol. The 1,4-cyclohexanedimethanol pathway includes an enzyme selected from a p-toluate monooxygenase, a p-hydroxymethyl benzoate reductase, a dihydroxymethyl benzene dehydrogenase, a p-methylbenzoyl-CoA synthetase, transferase and/or hydrolase, a p-toluate kinase, a phosphotrans-p-methylbenzoylase, a (p-methylbenzoyloxy)phosphonate reductase (dephosphorylating), a p-methylbenzyl alcohol dehydrogenase, a p-methylbenzoyl-CoA reductase, a p-toluate reductase, a p-methylbenzyl alcohol monooxygenase, a p-hydroxymethyl benzoyl-CoA synthetase, transferase and/or hydrolase, a p-hydroxymethyl benzoyl-CoA reductase, a p-hydroxymethyl benzoate kinase, a phosphotrans-p-hydroxymethylbenzoylase, and a (p-hydroxymethylbenzoyloxy)phosphonate reductase (dephosphorylating).

In one embodiment, the invention provides a non-naturally occurring microbial organism that includes a microbial organism having a 1,4-cyclohexanedimethanol pathway having at least one exogenous nucleic acid encoding a 1,4-cyclohexanedimethanol pathway enzyme expressed in a sufficient amount to produce 1,4-cyclohexanedimethanol. The 1,4-cyclohexanedimethanol pathway includes an enzyme set selected from the pathways shown in FIG. 1 and FIG. 2 and include (1) a p-toluate monooxygenase, a p-hydroxymethyl benzoate reductase, a dihydroxymethyl benzene dehydrogenase, and chemical reduction; (2) a p-toluate kinase, a (p-methylbenzoyloxy)phosphonate reductase (dephosphorylating), a p-methylbenzyl alcohol dehydrogenase, and a p-methylbenzyl alcohol monooxygenase, and chemical reduction; (3) a p-methylbenzoyl-CoA synthetase, transferase and/or hydrolase, a phosphotrans-p-methylbenzoylase, a (p-methylbenzoyloxy)phosphonate reductase (dephosphorylating), a p-methylbenzyl alcohol dehydrogenase, a p-methylbenzyl alcohol monooxygenase; and chemical reduction; (4) a p-methylbenzoyl-CoA synthetase, transferase and/or hydrolase, a p-methylbenzoyl-CoA reductase, a p-methylbenzyl alcohol dehydrogenase, a p-methylbenzyl alcohol monooxygenase, and chemical reduction; (5) a p-toluate reductase, a p-methylbenzyl alcohol dehydrogenase, and a p-methylbenzyl alcohol monooxygenase, and chemical reduction; (6) a p-toluate monooxygenase, a p-hydroxymethyl benzoyl-CoA synthetase, transferase and/or hydrolase, a p-hydroxymethyl benzoyl-CoA reductase, a dihydroxymethyl benzene dehdyrogenase, and chemical reduction; (7) a p-toluate monooxygenase, a p-hydroxymethyl benzoate kinase, a (p-hydroxymethylbenzoyloxy)phosphonate reductase (dephosphorylating), a dihydroxymethyl benzene dehdyrogenase, and chemical reduction; (8) a p-toluate monooxygenase, a p-hydroxymethyl benzoate kinase, a phosphotrans-p-hydroxymethylbenzoylase, a p-hydroxymethyl benzoyl-CoA reductase, a dihydroxymethyl benzene dehydrogenase, and chemical reduction; (9) a p-toluate monooxygenase, a p-hydroxymethyl benzoate kinase, a phosphotrans-p-hydroxymethylbenzoylase, a p-hydroxymethyl benzoyl-CoA reductase, a 4-hydroxymethyl cyclohex-1,5-diene carboxyl CoA reductase, a 4-hydroxymethyl cyclohex-1-ene carboxyl-CoA reductase, a 4-hydroxymethylcyclohexanoyl-CoA reductase, and a 4-hydroxymethyl cyclohexane carbaldehyde reductase; and (10) a p-toluate monooxygenase, a p-hydroxymethyl benzoyl-CoA synthetase, transferase and/or hydrolase, a p-hydroxymethyl benzoyl-CoA reductase, a 4-hydroxymethyl cyclohex-1,5-diene carboxyl CoA reductase, a 4-hydroxymethyl cyclohex-1-ene carboxyl-CoA reductase, a 4-hydroxymethylcyclohexanoyl-CoA reductase, and a 4-hydroxymethyl cyclohexane carbaldehyde reductase.

In another embodiment, the invention provides a method for producing 1,4-cyclohexanedimethanol that includes culturing such non-naturally occurring microbial organism having a 1,4-cyclohexanedimethanol pathway, under conditions and for a sufficient period of time to produce 1,4-cyclohexanedimethanol after reduction of dihydroxymethyl benzene.

As used herein, the term “non-naturally occurring” when used in reference to a microbial organism or microorganism of the invention is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes or proteins within a 1,4-cyclohexanedimethanol biosynthetic pathway.

A metabolic modification refers to a biochemical reaction that is altered from its naturally occurring state. Therefore, non-naturally occurring microorganisms can have genetic modifications to nucleic acids encoding metabolic polypeptides, or functional fragments thereof. Exemplary metabolic modifications are disclosed herein.

As used herein, the term “isolated” when used in reference to a microbial organism is intended to mean an organism that is substantially free of at least one component as the referenced microbial organism is found in nature. The term includes a microbial organism that is removed from some or all components as it is found in its natural environment. The term also includes a microbial organism that is removed from some or all components as the microbial organism is found in non-naturally occurring environments. Therefore, an isolated microbial organism is partly or completely separated from other substances as it is found in nature or as it is grown, stored or subsisted in non-naturally occurring environments. Specific examples of isolated microbial organisms include partially pure microbes, substantially pure microbes and microbes cultured in a medium that is non-naturally occurring.

As used herein, the terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

As used herein, the term “CoA” or “coenzyme A” is intended to mean an organic cofactor or prosthetic group (nonprotein portion of an enzyme) whose presence is required for the activity of many enzymes (the apoenzyme) to form an active enzyme system. Coenzyme A functions in certain condensing enzymes, acts in acetyl or other acyl group transfer and in fatty acid synthesis and oxidation, pyruvate oxidation and in other acetylation.

As used herein, the term “substantially anaerobic” when used in reference to a culture or growth condition is intended to mean that the amount of oxygen is less than about 10% of saturation for dissolved oxygen in liquid media. The term also is intended to include sealed chambers of liquid or solid medium maintained with an atmosphere of less than about 1% oxygen.

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid of the invention can utilize either or both a heterologous or homologous encoding nucleic acid.

It is understood that when more than one exogenous nucleic acid is included in a microbial organism that the more than one exogenous nucleic acids refers to the referenced encoding nucleic acid or biosynthetic activity, as discussed above. It is further understood, as disclosed herein, that such more than one exogenous nucleic acids can be introduced into the host microbial organism on separate nucleic acid molecules, on polycistronic nucleic acid molecules, or a combination thereof, and still be considered as more than one exogenous nucleic acid. For example, as disclosed herein a microbial organism can be engineered to express two or more exogenous nucleic acids encoding a desired pathway enzyme or protein. In the case where two exogenous nucleic acids encoding a desired activity are introduced into a host microbial organism, it is understood that the two exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two exogenous nucleic acids can be introduced into a host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host organism.

The non-naturally occurring microbal organisms of the invention can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

Those skilled in the art will understand that the genetic alterations, including metabolic modifications exemplified herein, are described with reference to a suitable host organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less that 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical product, those skilled in the art will understand that the orthologous gene harboring the metabolic activity to be introduced or disrupted is to be chosen for construction of the non-naturally occurring microorganism. An example of orthologs exhibiting separable activities is where distinct activities have been separated into distinct gene products between two or more species or within a single species. A specific example is the separation of elastase proteolysis and plasminogen proteolysis, two types of serine protease activity, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the non-naturally occurring microbial organisms of the invention having 1,4-cyclohexanedimethanol biosynthetic capability, those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced microorganism that encode an enzyme catalyzing a similar or substantially similar metabolic reaction, those skilled in the art also can utilize these evolutionally related genes.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

In some embodiments, the present invention provides a non-naturally occurring microbial organism that includes a microbial organism having a 1,4-cyclohexanedimethanol pathway that includes at least one exogenous nucleic acid encoding a 1,4-cyclohexanedimethanol pathway enzyme expressed in a sufficient amount to produce 1,4-cyclohexanedimethanol. The 1,4-cyclohexanedimethanol pathway includes an enzyme set selected from (1) a p-toluate monooxygenase, a p-hydroxymethyl benzoate reductase, a dihydroxymethyl benzene dehydrogenase, and chemical reduction; (2) a p-toluate kinase, a (p-methylbenzoyloxy)phosphonate reductase (dephosphorylating), a p-methylbenzyl alcohol dehydrogenase, a p-methylbenzyl alcohol monooxygenase, and chemical reduction; (3) a p-methylbenzoyl-CoA synthetase, transferase and/or hydrolase, a phosphotrans-p-methylbenzoylase, a (p-methylbenzoyloxy)phosphonate reductase (dephosphorylating), a p-methylbenzyl alcohol dehydrogenase, a p-methylbenzyl alcohol monooxygenase; and chemical reduction; (4) a p-methylbenzoyl-CoA synthetase, transferase and/or hydrolase, a p-methylbenzoyl-CoA reductase, a p-methylbenzyl alcohol dehydrogenase, a p-methylbenzyl alcohol monooxygenase, and chemical reduction; (5) a p-toluate reductase, a p-methylbenzyl alcohol dehydrogenase, a p-methylbenzyl alcohol monooxygenase, and chemical reduction; (6) a p-toluate monooxygenase, a p-hydroxymethyl benzoyl-CoA synthetase, transferase and/or hydrolase, p-hydroxymethyl benzoyl-CoA reductase, a dihydroxymethyl benzene dehydrogenase, and chemical reduction; (7) a p-toluate monooxygenase, a p-hydroxymethyl benzoate kinase, a (p-hydroxymethylbenzoyloxy)phosphonate reductase (dephosphorylating), a dihydroxymethyl benzene dehydrogenase, and chemical reduction; (8) a p-toluate monooxygenase, a p-hydroxymethyl benzoate kinase, a phosphotrans-p-hydroxymethylbenzoylase, a p-hydroxymethyl benzoyl-CoA reductase, a dihydroxymethyl benzene dehydrogenase, and chemical reduction; (9) a p-toluate monooxygenase, a p-hydroxymethyl benzoate kinase, a phosphotrans-p-hydroxymethylbenzoylase, a p-hydroxymethyl benzoyl-CoA reductase, a 4-hydroxymethyl cyclohex-1,5-diene carboxyl CoA reductase, a 4-hydroxymethyl cyclohex-1-ene carboxyl-CoA reductase, a 4-hydroxymethylcyclohexanoyl-CoA reductase, and a 4-hydroxymethyl cyclohexane carbaldehyde reductase; and (10) a p-toluate monooxygenase, a p-hydroxymethyl benzoyl-CoA synthetase, transferase and/or hydrolase, a p-hydroxymethyl benzoyl-CoA reductase, a 4-hydroxymethyl cyclohex-1,5-diene carboxyl CoA reductase, a 4-hydroxymethyl cyclohex-1-ene carboxyl-CoA reductase, a 4-hydroxymethylcyclohexanoyl-CoA reductase, and a 4-hydroxymethyl cyclohexane carbaldehyde reductase as shown in FIG. 1 and FIG. 2.

In some embodiments, a non-naturally occurring microbial organism of the invention can include two exogenous nucleic acids each encoding a 1,4-cyclohexanedimethanol pathway enzyme. In some embodiments, the non-naturally occurring microbial organism of the invention can include three exogenous nucleic acids each encoding a 1,4-cyclohexanedimethanol pathway enzyme. In some embodiments, the non-naturally occurring microbial organism of the invention includes four exogenous nucleic acids each encoding a 1,4-cyclohexanedimethanol pathway enzyme. In some such embodiments, the non-naturally occurring microbial organism of the invention can include, for example, four exogenous nucleic acids, each of which encodes an enzyme in enzyme set (1), (5), (6), or (7) of FIG. 1. In some embodiments, the non-naturally occurring microbial organism of the invention can include five exogenous nucleic acids each encoding a 1,4-cyclohexanedimethanol pathway enzyme. In some such embodiments, the non-naturally occurring microbial organism of the invention can include, for example, five exogenous nucleic acids each of which encodes an enzyme in enzyme set (2), (4), or (8) of FIG. 1. In some embodiments, the non-naturally occurring microbial organism of the invention includes six exogenous nucleic acids each encoding a 1,4-cyclohexanedimethanol pathway enzyme. In some such embodiments, the non-naturally occurring microbial organism of the invention can include, for example six exogenous nucleic acids, each of which encode enzyme in enzyme set (3) of FIG. 1. In some embodiments, the non-naturally occurring microbial organism of the invention includes seven exogenous nucleic acids each encoding a 1,4-cyclohexanedimethanol pathway enzyme. In some such embodiments, the non-naturally occurring microbial organism of the invention can include, for example seven exogenous nucleic acids, each of which encode enzyme in enzyme set (9) of FIGS. 1 and 2. In some embodiments, the non-naturally occurring microbial organism of the invention includes eighth exogenous nucleic acids each encoding a 1,4-cyclohexanedimethanol pathway enzyme. In some such embodiments, the non-naturally occurring microbial organism of the invention can include, for example eight exogenous nucleic acids, each of which encode enzyme in enzyme set (10) of FIGS. 1 and 2.

In some embodiments, the non-naturally occurring microbial organism of the invention includes at least one exogenous nucleic acid as a heterologous nucleic acid. In some embodiments, the non-naturally occurring microbial organism of the invention includes a non-naturally occurring microbial organism that is in a substantially anaerobic culture medium.

In an additional embodiment, the invention provides a non-naturally occurring microbial organism having a 1,4-cyclohexanedimethanol pathway, wherein the non-naturally occurring microbial organism comprises at least one exogenous nucleic acid encoding an enzyme or protein that converts a substrate to a product selected from the group consisting of p-toluate to p-hydroxymethylbenzoate, p-hydroxymethylbenzoate to p-hydroxymethylbenzaldehyde, p-hydroxymethylbenzaldehyde to dihydroxymethyl benzene, dihydroxymethyl benzene to 1,4-cyclohexanedimethanol, p-toluate to p-methylbenzoyloxyphosphonate, p-methylbenzoyloxyphosphonate to p-methylbenzaldehyde, p-methylbenzaldehyde to p-methylbenzyl alcohol, p-methylbenzyl alcohol to dihydroxymethyl benzene, p-toluate to p-methylbenzoyl-CoA, p-methylbenzoyl-CoA to p-methylbenzoyloxyphosphonate, p-methylbenzoyl-CoA to p-methylbenzaldehyde, p-toluate to p-methylbenzaldehyde, p-hydroxymethylbenzoate to p-hydroxymethylbenzoyloxy phosphonate, p-hydroxymethylbenzoyloxy phosphonate to p-hydroxybenzaldehyde, p-hydroxymethylbenzoyloxy phosphonate to p-hydroxymethylbenzoyl-CoA, p-hydroxymethylbenzoate to p-hydroxymethylbenzoyl-CoA, p-hydroxymethylbenzoyl-CoA to p-hydroxymethylbenzaldehyde, p-hydroxymethylbenzoyl-CoA to 4-hydroxymethylcyclohex-1-diene carboxyl-CoA, 4-hydroxymethylcyclohex-1,5-diene carboxyl-CoA to 4-hydroxymethylcyclohex-1-ene carboxyl-CoA, 4-hydroxymethylcyclohex-1-ene carboxyl-CoA to 4-hydroxymethylcycohexanoyl-CoA, 4-hydroxymethylcycohexanoyl-CoA to 4-hydroxymethylcyclohexane carbaldehyde, and 4-hydroxymethylcyclohexane carbaldehyde to 1,4-cyclohexanedimethanol. One skilled in the art will understand that these are merely exemplary and that any of the substrate-product pairs disclosed herein suitable to produce a desired product and for which an appropriate activity is available for the conversion of the substrate to the product can be readily determined by one skilled in the art based on the teachings herein. Thus, the invention provides a non-naturally occurring microbial organism containing at least one exogenous nucleic acid encoding an enzyme or protein, where the enzyme or protein converts the substrates and products of a 1,4-cyclohexanedimethanol pathway, such as that shown in FIG. 1.

While generally described herein as a microbial organism that contains a 1,4-cyclohexanedimethanol pathway, it is understood that the invention additionally provides a non-naturally occurring microbial organism comprising at least one exogenous nucleic acid encoding a 1,4-cyclohexanedimethanol pathway enzyme expressed in a sufficient amount to produce an intermediate of a 1,4-cyclohexanedimethanol pathway. For example, as disclosed herein, a 1,4-cyclohexanedimethanol pathway is exemplified in FIG. 1 and FIG. 2. Therefore, in addition to a microbial organism containing a 1,4-cyclohexanedimethanol pathway that produces 1,4-cyclohexane dimethanol, the invention additionally provides anon-naturally occurring microbial organism comprising at least one exogenous nucleic acid encoding a 1,4-cyclohexanedimethanol pathway enzyme, where the microbial organism produces a 1,4-cyclohexanedimethanol pathway intermediate, for example, p-toluate, p-hydroxymethylbenzoate, p-hydroxymethylbenzaldehyde, p-methylbenzoyl-CoA, p-methylbenzoyloxyphosphonate, p-methylbenzaldehyde, p-methylbenzyl alcohol, dihydroxymethyl benzene, p-hydroxymethylbenzoyloxyphosphonate, p-hydroxymethylbenzoyl-CoA, 4-hydroxymethylcycohex-1,5-diene carboxyl-CoA, 4-hydroxymethylcyclohex-1-ene carboxyl-CoA, 4-hydroxymethylcyclohexanoyl-CoA, and 4-hydroxymethylcyclohexane carbaldehyde.

It is understood that any of the pathways disclosed herein, as described in the Examples and exemplified in the pathways of FIGS. 1 and 2, can be utilized to generate a non-naturally occurring microbial organism that produces any pathway intermediate or product, as desired. As disclosed herein, such a microbial organism that produces an intermediate can be used in combination with another microbial organism expressing downstream pathway enzymes to produce a desired product. However, it is understood that a non-naturally occurring microbial organism that produces a 1,4-cyclohexanedimethanol pathway intermediate can be utilized to produce the intermediate as a desired product.

The invention is described herein with general reference to the metabolic reaction, reactant or product thereof, or with specific reference to one or more nucleic acids or genes encoding an enzyme associated with or catalyzing, or a protein associated with, the referenced metabolic reaction, reactant or product. Unless otherwise expressly stated herein, those skilled in the art will understand that reference to a reaction also constitutes reference to the reactants and products of the reaction. Similarly, unless otherwise expressly stated herein, reference to a reactant or product also references the reaction, and reference to any of these metabolic constituents also references the gene or genes encoding the enzymes that catalyze or proteins involved in the referenced reaction, reactant or product. Likewise, given the well known fields of metabolic biochemistry, enzymology and genomics, reference herein to a gene or encoding nucleic acid also constitutes a reference to the corresponding encoded enzyme and the reaction it catalyzes or a protein associated with the reaction as well as the reactants and products of the reaction.

The non-naturally occurring microbial organisms of the invention can be produced by introducing expressible nucleic acids encoding one or more of the enzymes or proteins participating in one or more 1,4-cyclohexanedimethanol biosynthetic pathways. Depending on the host microbial organism chosen for biosynthesis, nucleic acids for some or all of a particular 1,4-cyclohexanedimethanol biosynthetic pathway can be expressed. For example, if a chosen host is deficient in one or more enzymes or proteins for a desired biosynthetic pathway, then expressible nucleic acids for the deficient enzyme(s) or protein(s) are introduced into the host for subsequent exogenous expression. Alternatively, if the chosen host exhibits endogenous expression of some pathway genes, but is deficient in others, then an encoding nucleic acid is needed for the deficient enzyme(s) or protein(s) to achieve 1,4-cyclohexanedimethanol biosynthesis. Thus, a non-naturally occurring microbial organism of the invention can be produced by introducing exogenous enzyme or protein activities to obtain a desired biosynthetic pathway or a desired biosynthetic pathway can be obtained by introducing one or more exogenous enzyme or protein activities that, together with one or more endogenous enzymes or proteins, produces a desired product such as 1,4-cyclohexanedimethanol.

Host microbial organisms can be selected from, and the non-naturally occurring microbial organisms generated in, for example, bacteria, yeast, fungus or any of a variety of other microorganisms applicable to fermentation processes. Exemplary bacteria include species selected from Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Exemplary yeasts or fungi include species selected from Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, and the like. E. coli is a particularly useful host organism since it is a well characterized microbial organism suitable for genetic engineering. Other particularly useful host organisms include yeast such as Saccharomyces cerevisiae. It is understood that any suitable microbial host organism can be used to introduce metabolic and/or genetic modifications to produce a desired product.

Depending on the 1,4-cyclohexanedimethanol biosynthetic pathway constituents of a selected host microbial organism, the non-naturally occurring microbial organisms of the invention will include at least one exogenously expressed 1,4-cyclohexanedimethanol pathway-encoding nucleic acid and up to all encoding nucleic acids for one or more 1,4-cyclohexanedimethanol biosynthetic pathways. For example, 1,4-cyclohexanedimethanol biosynthesis can be established in a host deficient in a pathway enzyme or protein through exogenous expression of the corresponding encoding nucleic acid. In a host deficient in all enzymes or proteins of a 1,4-cyclohexanedimethanol pathway, exogenous expression of all enzyme or proteins in the pathway can be included, although it is understood that all enzymes or proteins of a pathway can be expressed even if the host contains at least one of the pathway enzymes or proteins. For example, exogenous expression of all enzymes or proteins in a pathway for production of 1,4-cyclohexanedimethanol can be included, such as a p-toluate monooxygenase, a p-hydroxymethyl benzoate reductase, a dihydroxymethyl benzene dehydrogenase, a p-methylbenzoyl-CoA synthetase, transferase and/or hydrolase, a p-toluate kinase, a phosphotrans-p-methylbenzoylase, a (p-methylbenzoyloxy)phosphonate reductase (dephosphorylating), a p-methylbenzyl alcohol dehydrogenase, a p-methylbenzoyl-CoA reductase, a p-toluate reductase, a p-methylbenzyl alcohol monooxygenase, a p-hydroxymethyl benzoyl-CoA synthetase, transferase and/or hydrolase, a p-hydroxymethyl benzoyl-CoA reductase, a p-hydroxymethyl benzoate kinase a phosphotrans-p-hydroxymethylbenzoylase, a (p-hydroxymethylbenzoyloxy) phosphonate reductase (dephosphorylating), a p-hydroxymethyl benzoyl-CoA reductase, a 4-hydroxymethyl cyclohex-1,5-diene carboxyl CoA reductase, a 4-hydroxymethyl cyclohex-1-ene carboxyl-CoA reductase, a 4-hydroxymethylcyclohexanoyl-CoA reductase, and a 4-hydroxymethyl cyclohexane carbaldehyde reductase.

Given the teachings and guidance provided herein, those skilled in the art will understand that the number of encoding nucleic acids to introduce in an expressible form will, at least, parallel the 1,4-cyclohexanedimethanol pathway deficiencies of the selected host microbial organism. Therefore, a non-naturally occurring microbial organism of the invention can have one, two, three, four, five, six, seven, eight up to all nucleic acids encoding the enzymes or proteins constituting a 1,4-cyclohexanedimethanol biosynthetic pathway disclosed herein. In some embodiments, the non-naturally occurring microbial organisms also can include other genetic modifications that facilitate or optimize 1,4-cyclohexanedimethanol biosynthesis or that confer other useful functions onto the host microbial organism. One such other functionality can include, for example, augmentation of the synthesis of one or more of the 1,4-cyclohexanedimethanol pathway precursors such as p-toluate, p-hydroxymethylbenzoate, p-hydroxymethylbenzaldehyde, p-methylbenzoyl-CoA, p-methylbenzoyloxyphosphonate, p-methylbenzaldehyde, p-methylbenzyl alcohol, dihydroxymethyl benzene, p-hydroxymethylbenzoyloxyphosphonate, p-hydroxymethylbenzoyl-CoA, 4-hydroxymethylcycohex-1,5-diene carboxyl-CoA, 4-hydroxymethylcyclohex-1-ene carboxyl-CoA, 4-hydroxymethylcyclohexanoyl-CoA, and 4-hydroxymethylcyclohexane carbaldehyde.

Generally, a host microbial organism is selected such that it produces the precursor of a 1,4-cyclohexanedimethanol pathway, either as a naturally produced molecule or as an engineered product that either provides de novo production of a desired precursor or increased production of a precursor naturally produced by the host microbial organism. For example, p-toluate, p-hydroxymethylbenzoate, p-hydroxymethylbenzaldehyde, p-methylbenzoyl-CoA, p-methylbenzoyloxyphosphonate, p-methylbenzaldehyde, p-methylbenzyl alcohol, dihydroxymethyl benzene, p-hydroxymethylbenzoyloxyphosphonate, p-hydroxymethylbenzoyl-CoA, 4-hydroxymethylcycohex-1,5-diene carboxyl-CoA, 4-hydroxymethylcyclohex-1-ene carboxyl-CoA, 4-hydroxymethylcyclohexanoyl-CoA, and 4-hydroxymethylcyclohexane carbaldehyde is produced naturally in a host organism such as E. coli. A host organism can be engineered to increase production of a precursor, as disclosed herein. In addition, a microbial organism that has been engineered to produce a desired precursor can be used as a host organism and further engineered to express enzymes or proteins of a 1,4-cyclohexanedimethanol pathway.

In some embodiments, a non-naturally occurring microbial organism of the invention is generated from a host that contains the enzymatic capability to synthesize 1,4-cyclohexanedimethanol. In this specific embodiment it can be useful to increase the synthesis or accumulation of a 1,4-cyclohexanedimethanol pathway product to, for example, drive 1,4-cyclohexanedimethanol pathway reactions toward 1,4-cyclohexanedimethanol production. Increased synthesis or accumulation can be accomplished by, for example, overexpression of nucleic acids encoding one or more of the above-described 1,4-cyclohexanedimethanol pathway enzymes or proteins. Over expression the enzyme or enzymes and/or protein or proteins of the 1,4-cyclohexanedimethanol pathway can occur, for example, through exogenous expression of the endogenous gene or genes, or through exogenous expression of the heterologous gene or genes. Therefore, naturally occurring organisms can be readily generated to be non-naturally occurring microbial organisms of the invention, for example, producing 1,4-cyclohexanedimethanol, through overexpression of one, two, three, four, five, six, seven, eight, that is, up to all nucleic acids encoding 1,4-cyclohexanedimethanol biosynthetic pathway enzymes or proteins. In addition, a non-naturally occurring organism can be generated by mutagenesis of an endogenous gene that results in an increase in activity of an enzyme in the 1,4-cyclohexanedimethanol biosynthetic pathway.

In particularly useful embodiments, exogenous expression of the encoding nucleic acids is employed. Exogenous expression confers the ability to custom tailor the expression and/or regulatory elements to the host and application to achieve a desired expression level that is controlled by the user. However, endogenous expression also can be utilized in other embodiments such as by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring microbial organism.

It is understood that, in methods of the invention, any of the one or more exogenous nucleic acids can be introduced into a microbial organism to produce a non-naturally occurring microbial organism of the invention. The nucleic acids can be introduced so as to confer, for example, a 1,4-cyclohexanedimethanol biosynthetic pathway onto the microbial organism. Alternatively, encoding nucleic acids can be introduced to produce an intermediate microbial organism having the biosynthetic capability to catalyze some of the required reactions to confer 1,4-cyclohexanedimethanol biosynthetic capability. For example, a non-naturally occurring microbial organism having a 1,4-cyclohexanedimethanol biosynthetic pathway can comprise at least two exogenous nucleic acids encoding desired enzymes or proteins, such as the combination of a p-toluate monooxygenase and a p-hydroxymethyl benzoate reductase, a p-toluate monooxygenase and a dihydroxymethyl benzene dehydrogenase, a p-hydroxymethyl benzoate reductase and a dihydroxymethyl benzene dehydrogenase, a p-toluate kinase and a (p-methylbenzoyloxy)phosphonate reductase (dephosphorylating), a p-toluate kinase and a p-methylbenzyl alcohol dehydrogenase, a p-toluate kinase and a p-methylbenzyl alcohol monooxygenase, a (p-methylbenzoyloxy)phosphonate reductase (dephosphorylating) and a p-methylbenzyl alcohol dehydrogenase, a (p-methylbenzoyloxy)phosphonate reductase (dephosphorylating) and a p-methylbenzyl alcohol monooxygenase, a p-methylbenzyl alcohol dehydrogenase and a p-methylbenzyl alcohol monooxygenase, a p-methylbenzoyl-CoA synthetase, transferase and/or hydrolase and a phosphotrans-p-methylbenzoylase, a p-methylbenzoyl-CoA synthetase, transferase and/or hydrolase (p-methylbenzoyloxy)phosphonate reductase (dephosphorylating), a p-methylbenzoyl-CoA synthetase, transferase and/or hydrolase and a p-methylbenzyl alcohol dehydrogenase, a p-methylbenzoyl-CoA synthetase, transferase and/or hydrolase and a p-methylbenzyl alcohol monooxygenase, a phosphotrans-p-methylbenzoylase and a (p-methylbenzoyloxy)phosphonate reductase (dephosphorylating), a phosphotrans-p-methylbenzoylase and a p-methylbenzyl alcohol dehydrogenase, a phosphotrans-p-methylbenzoylase and a p-methylbenzyl alcohol monooxygenase, a p-methylbenzoyl-CoA synthetase, transferase and/or hydrolase and a p-methylbenzoyl-CoA reductase, a p-methylbenzoyl-CoA reductase and a p-methylbenzyl alcohol dehydrogenase, a p-methylbenzoyl-CoA reductase and a p-methylbenzyl alcohol monooxygenase, a p-toluate reductase and a p-methylbenzyl alcohol dehydrogenase, and a p-toluate reductase and a p-methylbenzyl alcohol monooxygenase. Thus, it is understood that any combination of two or more enzymes or proteins of a biosynthetic pathway can be included in a non-naturally occurring microbial organism of the invention.

Similarly, it is understood that any combination of three or more enzymes or proteins of a biosynthetic pathway can be included in a non-naturally occurring microbial organism of the invention, for example, a p-toluate monooxygenase, a p-hydroxymethyl benzoate reductase, and a dihydroxymethyl benzene dehydrogenase, a p-toluate kinase, a (p-methylbenzoyloxy)phosphonate reductase (dephosphorylating), and a p-methylbenzyl alcohol dehydrogenase, a p-toluate kinase, a (p-methylbenzoyloxy)phosphonate reductase (dephosphorylating), and a p-methylbenzyl alcohol monooxygenase, p-toluate reductase, p-methylbenzyl alcohol dehydrogenase, p-methylbenzyl alcohol monooxygenase, p-methylbenzoyl-CoA synthetase, transferase and/or hydrolase, p-methylbenzoyl-CoA reductase, p-methylbenzyl alcohol dehydrogenase, and so forth, as desired, so long as the combination of enzymes and/or proteins of the desired biosynthetic pathway results in production of the corresponding desired product. Similarly, any combination of four, five, six, seven, eight or more enzymes or proteins of a biosynthetic pathway as disclosed herein can be included in a non-naturally occurring microbial organism of the invention, as desired, so long as the combination of enzymes and/or proteins of the desired biosynthetic pathway results in production of the corresponding desired product.

In addition to the biosynthesis of 1,4-cyclohexanedimethanol as described herein, the non-naturally occurring microbial organisms and methods of the invention also can be utilized in various combinations with each other and with other microbial organisms and methods well known in the art to achieve product biosynthesis by other routes. For example, one alternative to produce 1,4-cyclohexanedimethanol other than use of the 1,4-cyclohexanedimethanol producers is through addition of another microbial organism capable of converting a 1,4-cyclohexanedimethanol pathway intermediate to 1,4-cyclohexane dimethanol. One such procedure includes, for example, the fermentation of a microbial organism that produces a 1,4-cyclohexanedimethanol pathway intermediate. The 1,4-cyclohexanedimethanol pathway intermediate can then be used as a substrate for a second microbial organism that converts the 1,4-cyclohexanedimethanol pathway intermediate to 1,4-cyclohexanedimethanol. The 1,4-cyclohexanedimethanol pathway intermediate can be added directly to another culture of the second organism or the original culture of the 1,4-cyclohexanedimethanol pathway intermediate producers can be depleted of these microbial organisms by, for example, cell separation, and then subsequent addition of the second organism to the fermentation broth can be utilized to produce the final product without intermediate purification steps.

In other embodiments, the non-naturally occurring microbial organisms and methods of the invention can be assembled in a wide variety of subpathways to achieve biosynthesis of, for example, 1,4-cyclohexanedimethanol. In these embodiments, biosynthetic pathways for a desired product of the invention can be segregated into different microbial organisms, and the different microbial organisms can be co-cultured to produce the final product. In such a biosynthetic scheme, the product of one microbial organism is the substrate for a second microbial organism until the final product is synthesized. For example, the biosynthesis of 1,4-cyclohexanedimethanol can be accomplished by constructing a microbial organism that contains biosynthetic pathways for conversion of one pathway intermediate to another pathway intermediate or the product. Alternatively, 1,4-cyclohexanedimethanol also can be biosynthetically produced from microbial organisms through co-culture or co-fermentation using two organisms in the same vessel, where the first microbial organism produces a 1,4-cyclohexanedimethanol intermediate and the second microbial organism converts the intermediate to 1,4-cyclohexane dimethanol.

Given the teachings and guidance provided herein, those skilled in the art will understand that a wide variety of combinations and permutations exist for the non-naturally occurring microbial organisms and methods of the invention together with other microbial organisms, with the co-culture of other non-naturally occurring microbial organisms having subpathways and with combinations of other chemical and/or biochemical procedures well known in the art to produce 1,4-cyclohexanedimethanol.

Sources of encoding nucleic acids for a 1,4-cyclohexanedimethanol pathway enzyme or protein can include, for example, any species where the encoded gene product is capable of catalyzing the referenced reaction. Such species include both prokaryotic and eukaryotic organisms including, but not limited to, bacteria, including archaea and eubacteria, and eukaryotes, including yeast, plant, insect, animal, and mammal, including human. Exemplary species for such sources include, for example, Escherichia coli, Thauera aromatica, Rhodopseudomonas palustris, Geobacter metallireducens, various Clostridia, as well as other exemplary species disclosed herein or available as source organisms for corresponding genes. However, with the complete genome sequence available for now more than 550 species (with more than half of these available on public databases such as the NCBI), including 395 microorganism genomes and a variety of yeast, fungi, plant, and mammalian genomes, the identification of genes encoding the requisite 1,4-cyclohexanedimethanol biosynthetic activity for one or more genes in related or distant species, including for example, homologues, orthologs, paralogs and nonorthologous gene displacements of known genes, and the interchange of genetic alterations between organisms is routine and well known in the art. Accordingly, the metabolic alterations allowing biosynthesis of 1,4-cyclohexanedimethanol described herein with reference to a particular organism such as E. coli can be readily applied to other microorganisms, including prokaryotic and eukaryotic organisms alike. Given the teachings and guidance provided herein, those skilled in the art will know that a metabolic alteration exemplified in one organism can be applied equally to other organisms.

In some instances, such as when an alternative 1,4-cyclohexanedimethanol biosynthetic pathway exists in an unrelated species, 1,4-cyclohexanedimethanol biosynthesis can be conferred onto the host species by, for example, exogenous expression of a paralog or paralogs from the unrelated species that catalyzes a similar, yet non-identical metabolic reaction to replace the referenced reaction. Because certain differences among metabolic networks exist between different organisms, those skilled in the art will understand that the actual gene usage between different organisms may differ. However, given the teachings and guidance provided herein, those skilled in the art also will understand that the teachings and methods of the invention can be applied to all microbial organisms using the cognate metabolic alterations to those exemplified herein to construct a microbial organism in a species of interest that will synthesize 1,4-cyclohexanedimethanol.

Methods for constructing and testing the expression levels of a non-naturally occurring 1,4-cyclohexanedimethanol-producing host can be performed, for example, by recombinant and detection methods well known in the art. Such methods can be found described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999).

Exogenous nucleic acid sequences involved in a pathway for production of 1,4-cyclohexanedimethanol can be introduced stably or transiently into a host cell using techniques well known in the art including, but not limited to, conjugation, electroporation, chemical transformation, transduction, transfection, and ultrasound transformation. For exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

An expression vector or vectors can be constructed to include one or more 1,4-cyclohexanedimethanol biosynthetic pathway encoding nucleic acids as exemplified herein operably linked to expression control sequences functional in the host organism. Expression vectors applicable for use in the microbial host organisms of the invention include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

In some embodiments, the present invention provides a method for producing 1,4-cyclohexanedimethanol that includes culturing a non-naturally occurring microbial organism having a 1,4-cyclohexanedimethanol pathway. The pathway includes at least one exogenous nucleic acid encoding a 1,4-cyclohexanedimethanol pathway enzyme expressed in a sufficient amount to produce 1,4-cyclohexanedimethanol, under conditions and for a sufficient period of time to produce 1,4-cyclohexanedimethanol, said 1,4-cyclohexanedimethanol pathway comprising an enzyme set selected from (1) a p-toluate monooxygenase, a p-hydroxymethyl benzoate reductase, a dihydroxymethyl benzene dehydrogenase, and chemical reduction; (2) a p-toluate kinase, a (p-methylbenzoyloxy)phosphonate reductase (dephosphorylating), a p-methylbenzyl alcohol dehydrogenase, and a p-methylbenzyl alcohol monooxygenase, and chemical reduction; (3) a p-methylbenzoyl-CoA synthetase, transferase and/or hydrolase, a phosphotrans-p-methylbenzoylase, a (p-methylbenzoyloxy)phosphonate reductase (dephosphorylating), a p-methylbenzyl alcohol dehydrogenase, and a p-methylbenzyl alcohol monooxygenase; and chemical reduction; (4) a p-methylbenzoyl-CoA synthetase, transferase and/or hydrolase, a p-methylbenzoyl-CoA reductase, a p-methylbenzyl alcohol dehydrogenase, and a p-methylbenzyl alcohol monooxygenase, and chemical reduction; and (5) a p-toluate reductase, a p-methylbenzyl alcohol dehydrogenase, and a p-methylbenzyl alcohol monooxygenase, and chemical reduction; (6) a p-toluate monooxygenase, a p-hydroxymethyl benzoyl-CoA synthetase, transferase and/or hydrolase, a p-hydroxymethyl benzoyl-CoA reductase, a dihydroxymethyl benzene dehydrogenase, and chemical reduction; (7) a p-toluate monooxygenase, a p-hydroxymethyl benzoate kinase, a (p-hydroxymethylbenzoyloxy)phosphonate reductase (dephosphorylating), a dihydroxymethyl benzene dehydrogenase, and chemical reduction; (8) a p-toluate monooxygenase, a p-hydroxymethyl benzoate kinase, a phosphotrans-p-hydroxymethylbenzoylase, a p-hydroxymethyl benzoyl-CoA reductase, a dihydroxymethyl benzene dehydrogenase, and chemical reduction; (9) a p-toluate monooxygenase, a p-hydroxymethyl benzoate kinase, a phosphotrans-p-hydroxymethylbenzoylase, a p-hydroxymethyl benzoyl-CoA reductase, a 4-hydroxymethyl cyclohex-1,5-diene carboxyl CoA reductase, a 4-hydroxymethyl cyclohex-1-ene carboxyl-CoA reductase, a 4-hydroxymethylcyclohexanoyl-CoA reductase, and a 4-hydroxymethyl cyclohexane carbaldehyde reductase; and (10) a p-toluate monooxygenase, a p-hydroxymethyl benzoyl-CoA synthetase, transferase and/or hydrolase, a p-hydroxymethyl benzoyl-CoA reductase, a 4-hydroxymethyl cyclohex-1,5-diene carboxyl CoA reductase, a 4-hydroxymethyl cyclohex-1-ene carboxyl-CoA reductase, a 4-hydroxymethylcyclohexanoyl-CoA reductase, and a 4-hydroxymethyl cyclohexane carbaldehyde reductase. In some embodiments, the method of the invention includes at least one exogenous nucleic acid is a heterologous nucleic acid. In some embodiments, the method of the invention includes a non-naturally occurring microbial organism that is in a substantially anaerobic culture medium.

In some embodiments, a method of the invention can include non-naturally occurring organisms having two exogenous nucleic acids each encoding a 1,4-cyclohexanedimethanol pathway enzyme. In some embodiments, a method of the invention can include non-naturally occurring organisms having three exogenous nucleic acids each encoding a 1,4-cyclohexanedimethanol pathway enzyme. In some embodiments, a method of the invention can include non-naturally occurring organisms having four exogenous nucleic acids each encoding a 1,4-cyclohexanedimethanol pathway enzyme. In some such embodiments, the non-naturally occurring microbial organism can include, for example, four exogenous nucleic acids, each of which encodes an enzyme in enzyme set (1), (5), (6), or (7) of FIG. 1. In some embodiments, the method of the invention can include non-naturally occurring microbial organisms having five exogenous nucleic acids each encoding a 1,4-cyclohexanedimethanol pathway enzyme. In some such embodiments, the non-naturally occurring microbial organism can include, for example, five exogenous nucleic acids each of which encodes an enzyme in enzyme set (2), (4) or (8) of FIG. 1. In some embodiments, the method of the invention includes a non-naturally occurring microbial organism having six exogenous nucleic acids each encoding a 1,4-cyclohexanedimethanol pathway enzyme. In some such embodiments, the non-naturally occurring microbial organism can include, for example six exogenous nucleic acids, each of which encode enzyme in enzyme set (3) of FIG. 1. In some embodiments, the method of the invention includes a non-naturally occurring microbial organism having seven exogenous nucleic acids each encoding a 1,4-cyclohexanedimethanol pathway enzyme. In some such embodiments, the non-naturally occurring microbial organism can include, for example seven exogenous nucleic acids, each of which encode enzyme in enzyme set (9) of FIGS. 1 and 2. In some embodiments, the method of the invention includes a non-naturally occurring microbial organism having eight exogenous nucleic acids each encoding a 1,4-cyclohexanedimethanol pathway enzyme. In some such embodiments, the non-naturally occurring microbial organism can include, for example eight exogenous nucleic acids, each of which encode enzyme in enzyme set (10) of FIGS. 1 and 2.

Suitable purification and/or assays to test for the production of 1,4-cyclohexanedimethanol can be performed using well known methods. Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid (chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the exogenous DNA sequences can also be assayed using methods well known in the art

The 1,4-cyclohexanedimethanol can be separated from other components in the culture using a variety of methods well known in the art. Such separation methods include, for example, extraction procedures as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, and ultrafiltration. All of the above methods are well known in the art.

Any of the non-naturally occurring microbial organisms described herein can be cultured to produce and/or secrete the biosynthetic products of the invention. For example, the 1,4-cyclohexanedimethanol producers can be cultured for the biosynthetic production of 1,4-cyclohexane dimethanol.

For the production of 1,4-cyclohexanedimethanol, the recombinant strains are cultured in a medium with carbon source and other essential nutrients. It is sometimes desirable and can be highly desirable to maintain anaerobic conditions in the fermenter to reduce the cost of the overall process. Such conditions can be obtained, for example, by first sparging the medium with nitrogen and then sealing the flasks with a septum and crimp-cap. For strains where growth is not observed anaerobically, microaerobic or substantially anaerobic conditions can be applied by perforating the septum with a small hole for limited aeration. Exemplary anaerobic conditions have been described previously and are well-known in the art. Exemplary aerobic and anaerobic conditions are described, for example, in United State publication 2009/0047719, filed Aug. 10, 2007. Fermentations can be performed in a batch, fed-batch or continuous manner, as disclosed herein.

If desired, the pH of the medium can be maintained at a desired pH, in particular neutral pH, such as a pH of around 7 by addition of a base, such as NaOH or other bases, or acid, as needed to maintain the culture medium at a desirable pH. The growth rate can be determined by measuring optical density using a spectrophotometer (600 nm), and the glucose uptake rate by monitoring carbon source depletion over time.

The growth medium can include, for example, any carbohydrate source which can supply a source of carbon to the non-naturally occurring microorganism. Such sources include, for example, sugars such as glucose, xylose, arabinose, galactose, mannose, fructose, sucrose and starch. Other sources of carbohydrate include, for example, renewable feedstocks and biomass. Exemplary types of biomasses that can be used as feedstocks in the methods of the invention include cellulosic biomass, hemicellulosic biomass and lignin feedstocks or portions of feedstocks. Such biomass feedstocks contain, for example, carbohydrate substrates useful as carbon sources such as glucose, xylose, arabinose, galactose, mannose, fructose and starch. Given the teachings and guidance provided herein, those skilled in the art will understand that renewable feedstocks and biomass other than those exemplified above also can be used for culturing the microbial organisms of the invention for the production of 1,4-cyclohexane dimethanol.

In addition to renewable feedstocks such as those exemplified above, the 1,4-cyclohexanedimethanol microbial organisms of the invention also can be modified for growth on syngas as its source of carbon. In this specific embodiment, one or more proteins or enzymes are expressed in the 1,4-cyclohexanedimethanol producing organisms to provide a metabolic pathway for utilization of syngas or other gaseous carbon source.

Synthesis gas, also known as syngas or producer gas, is the major product of gasification of coal and of carbonaceous materials such as biomass materials, including agricultural crops and residues. Syngas is a mixture primarily of H₂ and CO and can be obtained from the gasification of any organic feedstock, including but not limited to coal, coal oil, natural gas, biomass, and waste organic matter. Gasification is generally carried out under a high fuel to oxygen ratio. Although largely H₂ and CO, syngas can also include CO₂ and other gases in smaller quantities. Thus, synthesis gas provides a cost effective source of gaseous carbon such as CO and, additionally, CO₂.

The Wood-Ljungdahl pathway catalyzes the conversion of CO and H₂ to acetyl-CoA and other products such as acetate. Organisms capable of utilizing CO and syngas also generally have the capability of utilizing CO₂ and CO₂/H₂ mixtures through the same basic set of enzymes and transformations encompassed by the Wood-Ljungdahl pathway. H₂-dependent conversion of CO₂ to acetate by microorganisms was recognized long before it was revealed that CO also could be used by the same organisms and that the same pathways were involved. Many acetogens have been shown to grow in the presence of CO₂ and produce compounds such as acetate as long as hydrogen is present to supply the necessary reducing equivalents (see for example, Drake, Acetogenesis, pp. 3-60 Chapman and Hall, New York, (1994)). This can be summarized by the following equation:

2CO₂+4H₂+nADP+nPi→CH₃COOH+2H₂O+nATP

Hence, non-naturally occurring microorganisms possessing the Wood-Ljungdahl pathway can utilize CO₂ and H₂ mixtures as well for the production of acetyl-CoA and other desired products.

The Wood-Ljungdahl pathway is well known in the art and consists of 12 reactions which can be separated into two branches: (1) methyl branch and (2) carbonyl branch. The methyl branch converts syngas to methyl-tetrahydrofolate (methyl-THF) whereas the carbonyl branch converts methyl-THF to acetyl-CoA. The reactions in the methyl branch are catalyzed in order by the following enzymes or proteins: ferredoxin oxidoreductase, formate dehydrogenase, formyltetrahydrofolate synthetase, methenyltetrahydrofolate cyclodehydratase, methylenetetrahydrofolate dehydrogenase and methylenetetrahydrofolate reductase. The reactions in the carbonyl branch are catalyzed in order by the following enzymes or proteins: methyltetrahydrofolate:corrinoid protein methyltransferase (for example, AcsE), corrinoid iron-sulfur protein, nickel-protein assembly protein (for example, AcsF), ferredoxin, acetyl-CoA synthase, carbon monoxide dehydrogenase and nickel-protein assembly protein (for example, CooC). Following the teachings and guidance provided herein for introducing a sufficient number of encoding nucleic acids to generate a 1,4-cyclohexanedimethanol pathway, those skilled in the art will understand that the same engineering design also can be performed with respect to introducing at least the nucleic acids encoding the Wood-Ljungdahl enzymes or proteins absent in the host organism. Therefore, introduction of one or more encoding nucleic acids into the microbial organisms of the invention such that the modified organism contains the complete Wood-Ljungdahl pathway will confer syngas utilization ability.

Additionally, the reductive (reverse) tricarboxylic acid cycle is and/or hydrogenase activities can also be used for the conversion of CO, CO₂ and/or H₂ to acetyl-CoA and other products such as acetate. Organisms capable of fixing carbon via the reductive TCA pathway can utilize one or more of the following enzymes: ATP citrate-lyase, citrate lyase, aconitase, isocitrate dehydrogenase, alpha-ketoglutarate:ferredoxin oxidoreductase, succinyl-CoA synthetase, succinyl-CoA transferase, fumarate reductase, fumarase, malate dehydrogenase, NAD(P)H:ferredoxin oxidoreductase, carbon monoxide dehydrogenase, and hydrogenase. Specifically, the reducing equivalents extracted from CO and/or H₂ by carbon monoxide dehydrogenase and hydrogenase are utilized to fix CO₂ via the reductive TCA cycle into acetyl-CoA or acetate. Acetate can be converted to acetyl-CoA by enzymes such as acetyl-CoA transferase, acetate kinase/phosphotransacetylase, and acetyl-CoA synthetase. Acetyl-CoA can be converted to the 1,4-cyclohexanedimethanol precursors, glyceraldehyde-3-phosphate, phosphoenolpyruvate, and pyruvate, by pyruvate:ferredoxin oxidoreductase and the enzymes of gluconeogenesis. Following the teachings and guidance provided herein for introducing a sufficient number of encoding nucleic acids to generate a 1,4-cyclohexanedimethanol pathway, those skilled in the art will understand that the same engineering design also can be performed with respect to introducing at least the nucleic acids encoding the reductive TCA pathway enzymes or proteins absent in the host organism. Therefore, introduction of one or more encoding nucleic acids into the microbial organisms of the invention such that the modified organism contains the complete reductive TCA pathway will confer syngas utilization ability.

Accordingly, given the teachings and guidance provided herein, those skilled in the art will understand that a non-naturally occurring microbial organism can be produced that secretes the biosynthesized compounds of the invention when grown on a carbon source such as a carbohydrate. Such compounds include, for example, 1,4-cyclohexanedimethanol and any of the intermediate metabolites in the 1,4-cyclohexanedimethanol pathway. All that is required is to engineer in one or more of the required enzyme or protein activities to achieve biosynthesis of the desired compound or intermediate including, for example, inclusion of some or all of the 1,4-cyclohexanedimethanol biosynthetic pathways. Accordingly, the invention provides a non-naturally occurring microbial organism that produces and/or secretes 1,4-cyclohexanedimethanol when grown on a carbohydrate or other carbon source and produces and/or secretes any of the intermediate metabolites shown in the 1,4-cyclohexanedimethanol pathway when grown on a carbohydrate or other carbon source. The 1,4-cyclohexanedimethanol producing microbial organisms of the invention can initiate synthesis from an intermediate, for example, p-toluate, p-hydroxymethylbenzoate, p-hydroxymethylbenzaldehyde, p-methylbenzoyl-CoA, p-methylbenzoyloxyphosphonate, p-methylbenzaldehyde, p-methylbenzyl alcohol, dihydroxymethyl benzene, p-hydroxymethylbenzoyloxyphosphonate, p-hydroxymethylbenzoyl-CoA, 4-hydroxymethylcyclohex-1,5-diene carboxyl-CoA, 4-hydroxymethylcyclohex-1-ene carboxyl-CoA, 4-hydroxymethylcyclohexanoyl-CoA, and 4-hydroxymethylcyclohexane carbaldehyde.

The non-naturally occurring microbial organisms of the invention are constructed using methods well known in the art as exemplified herein to exogenously express at least one nucleic acid encoding a 1,4-cyclohexanedimethanol pathway enzyme or protein in sufficient amounts to produce 1,4-cyclohexanedimethanol. It is understood that the microbial organisms of the invention are cultured under conditions sufficient to produce 1,4-cyclohexanedimethanol. Following the teachings and guidance provided herein, the non-naturally occurring microbial organisms of the invention can achieve biosynthesis of 1,4-cyclohexanedimethanol resulting in intracellular concentrations between about 0.1-200 mM or more. Generally, the intracellular concentration of 1,4-cyclohexanedimethanol is between about 3-150 mM, particularly between about 5-125 mM and more particularly between about 8-100 mM, including about 10 mM, 20 mM, 50 mM, 80 mM, or more. Intracellular concentrations between and above each of these exemplary ranges also can be achieved from the non-naturally occurring microbial organisms of the invention.

In some embodiments, culture conditions include anaerobic or substantially anaerobic growth or maintenance conditions. Exemplary anaerobic conditions have been described previously and are well known in the art. Exemplary anaerobic conditions for fermentation processes are described herein and are described, for example, in U.S. publication 2009/0047719, filed Aug. 10, 2007. Any of these conditions can be employed with the non-naturally occurring microbial organisms as well as other anaerobic conditions well known in the art. Under such anaerobic or substantially anaerobic conditions, the 1,4-cyclohexanedimethanol producers can synthesize 1,4-cyclohexanedimethanol at intracellular concentrations of 5-10 mM or more as well as all other concentrations exemplified herein. It is understood that, even though the above description refers to intracellular concentrations, 1,4-cyclohexanedimethanol producing microbial organisms can produce 1,4-cyclohexanedimethanol intracellularly and/or secrete the product into the culture medium.

In addition to the culturing and fermentation conditions disclosed herein, growth condition for achieving biosynthesis of 1,4-cyclohexanedimethanol can include the addition of an osmoprotectant to the culturing conditions. In certain embodiments, the non-naturally occurring microbial organisms of the invention can be sustained, cultured or fermented as described herein in the presence of an osmoprotectant. Briefly, an osmoprotectant refers to a compound that acts as an osmolyte and helps a microbial organism as described herein survive osmotic stress. Osmoprotectants include, but are not limited to, betaines, amino acids, and the sugar trehalose. Non-limiting examples of such are glycine betaine, praline betaine, dimethylthetin, dimethyl slfonioproprionate, 3-dimethylsulfonio-2-methylproprionate, pipecolic acid, dimethylsulfonioacetate, choline, L-carnitine and ectoine. In one aspect, the osmoprotectant is glycine betaine. It is understood to one of ordinary skill in the art that the amount and type of osmoprotectant suitable for protecting a microbial organism described herein from osmotic stress will depend on the microbial organism used. The amount of osmoprotectant in the culturing conditions can be, for example, no more than about 0.1 mM, no more than about 0.5 mM, no more than about 1.0 mM, no more than about 1.5 mM, no more than about 2.0 mM, no more than about 2.5 mM, no more than about 3.0 mM, no more than about 5.0 mM, no more than about 7.0 mM, no more than about 10 mM, no more than about 50 mM, no more than about 100 mM or no more than about 500 mM.

The culture conditions can include, for example, liquid culture procedures as well as fermentation and other large scale culture procedures. As described herein, particularly useful yields of the biosynthetic products of the invention can be obtained under anaerobic or substantially anaerobic culture conditions.

As described herein, one exemplary growth condition for achieving biosynthesis of 1,4-cyclohexanedimethanol includes anaerobic culture or fermentation conditions. In certain embodiments, the non-naturally occurring microbial organisms of the invention can be sustained, cultured or fermented under anaerobic or substantially anaerobic conditions. Briefly, anaerobic conditions refers to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases.

The culture conditions described herein can be scaled up and grown continuously for manufacturing of 1,4-cyclohexane dimethanol. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of 1,4-cyclohexane dimethanol. Generally, and as with non-continuous culture procedures, the continuous and/or near-continuous production of 1,4-cyclohexanedimethanol will include culturing a non-naturally occurring 1,4-cyclohexanedimethanol producing organism of the invention in sufficient nutrients and medium to sustain and/or nearly sustain growth in an exponential phase. Continuous culture under such conditions can be include, for example, growth for 1 day, 2, 3, 4, 5, 6 or 7 days or more. Additionally, continuous culture can include longer time periods of 1 week, 2, 3, 4 or 5 or more weeks and up to several months. Alternatively, organism, of the invention can be cultured for hours, if suitable for a particular application. It is to be understood that the continuous and/or near-continuous culture conditions also can include all time intervals in between these exemplary periods. It is further understood that the time of culturing the microbial organism of the invention is for a sufficient period of time to produce a sufficient amount of product for a desired purpose.

Fermentation procedures are well known in the art. Briefly, fermentation for the biosynthetic production of 1,4-cyclohexanedimethanol can be utilized in, for example, fed-batch fermentation and batch separation; fed-batch fermentation and continuous separation, or continuous fermentation and continuous separation. Examples of batch and continuous fermentation procedures are well known in the art.

In addition to the above fermentation procedures using the 1,4-cyclohexanedimethanol producers of the invention for continuous production of substantial quantities of 1,4-cyclohexane dimethanol, the 1,4-cyclohexanedimethanol producers also can be, for example, simultaneously subjected to chemical synthesis procedures to convert the product to other compounds or the product can be separated from the fermentation culture and sequentially subjected to chemical conversion to convert the product to other compounds, if desired.

To generate better producers, metabolic modeling can be utilized to optimize growth conditions. Modeling can also be used to design gene knockouts that additionally optimize utilization of the pathway (see, for example, U.S. patent publications US 2002/0012939, US 2003/0224363, US 2004/0029149, US 2004/0072723, US 2003/0059792, US 2002/0168654 and US 2004/0009466, and U.S. Pat. No. 7,127,379). Modeling analysis allows reliable predictions of the effects on cell growth of shifting the metabolism towards more efficient production of 1,4-cyclohexane dimethanol.

One computational method for identifying and designing metabolic alterations favoring biosynthesis of a desired product is the OptKnock computational framework (Burgard et al., Biotechnol. Bioeng. 84:647-657 (2003)). OptKnock is a metabolic modeling and simulation program that suggests gene deletion or disruption strategies that result in genetically stable microorganisms which overproduce the target product. Specifically, the framework examines the complete metabolic and/or biochemical network of a microorganism in order to suggest genetic manipulations that force the desired biochemical to become an obligatory byproduct of cell growth. By coupling biochemical production with cell growth through strategically placed gene deletions or other functional gene disruption, the growth selection pressures imposed on the engineered strains after long periods of time in a bioreactor lead to improvements in performance as a result of the compulsory growth-coupled biochemical production. Lastly, when gene deletions are constructed there is a negligible possibility of the designed strains reverting to their wild-type states because the genes selected by OptKnock are to be completely removed from the genome. Therefore, this computational methodology can be used to either identify alternative pathways that lead to biosynthesis of a desired product or used in connection with the non-naturally occurring microbial organisms for further optimization of biosynthesis of a desired product.

Briefly, OptKnock is a term used herein to refer to a computational method and system for modeling cellular metabolism. The OptKnock program relates to a framework of models and methods that incorporate particular constraints into flux balance analysis (FBA) models. These constraints include, for example, qualitative kinetic information, qualitative regulatory information, and/or DNA microarray experimental data. OptKnock also computes solutions to various metabolic problems by, for example, tightening the flux boundaries derived through flux balance models and subsequently probing the performance limits of metabolic networks in the presence of gene additions or deletions. OptKnock computational framework allows the construction of model formulations that allow an effective query of the performance limits of metabolic networks and provides methods for solving the resulting mixed-integer linear programming problems. The metabolic modeling and simulation methods referred to herein as OptKnock are described in, for example, U.S. publication 2002/0168654, filed Jan. 10, 2002, in International Patent No. PCT/US02/00660, filed Jan. 10, 2002, and U.S. publication 2009/0047719, filed Aug. 10, 2007.

Another computational method for identifying and designing metabolic alterations favoring biosynthetic production of a product is a metabolic modeling and simulation system termed SimPheny®. This computational method and system is described in, for example, U.S. publication 2003/0233218, filed Jun. 14, 2002, and in International Patent Application No. PCT/US03/18838, filed Jun. 13, 2003. SimPheny® is a computational system that can be used to produce a network model in silico and to simulate the flux of mass, energy or charge through the chemical reactions of a biological system to define a solution space that contains any and all possible functionalities of the chemical reactions in the system, thereby determining a range of allowed activities for the biological system. This approach is referred to as constraints-based modeling because the solution space is defined by constraints such as the known stoichiometry of the included reactions as well as reaction thermodynamic and capacity constraints associated with maximum fluxes through reactions. The space defined by these constraints can be interrogated to determine the phenotypic capabilities and behavior of the biological system or of its biochemical components.

These computational approaches are consistent with biological realities because biological systems are flexible and can reach the same result in many different ways. Biological systems are designed through evolutionary mechanisms that have been restricted by fundamental constraints that all living systems must face. Therefore, constraints-based modeling strategy embraces these general realities. Further, the ability to continuously impose further restrictions on a network model via the tightening of constraints results in a reduction in the size of the solution space, thereby enhancing the precision with which physiological performance or phenotype can be predicted.

Given the teachings and guidance provided herein, those skilled in the art will be able to apply various computational frameworks for metabolic modeling and simulation to design and implement biosynthesis of a desired compound in host microbial organisms. Such metabolic modeling and simulation methods include, for example, the computational systems exemplified above as SimPheny® and OptKnock. For illustration of the invention, some methods are described herein with reference to the OptKnock computation framework for modeling and simulation. Those skilled in the art will know how to apply the identification, design and implementation of the metabolic alterations using OptKnock to any of such other metabolic modeling and simulation computational frameworks and methods well known in the art.

The methods described above will provide one set of metabolic reactions to disrupt. Elimination of each reaction within the set or metabolic modification can result in a desired product as an obligatory product during the growth phase of the organism. Because the reactior are known, a solution to the bilevel OptKnock problem also will provide the associated gene or genes encoding one or more enzymes that catalyze each reaction within the set of reactions. Identification of a set of reactions and their corresponding genes encoding the enzymes participating in each reaction is generally an automated process, accomplished through correlation of the reactions with a reaction database having a relationship between enzymes and encoding genes.

Once identified, the set of reactions that are to be disrupted in order to achieve production of a desired product are implemented in the target cell or organism by functional disruption of at least one gene encoding each metabolic reaction within the set. One particularly useful means to achieve functional disruption of the reaction set is by deletion of each encoding gene. However, in some instances, it can be beneficial to disrupt the reaction by other genetic aberrations including, for example, mutation, deletion of regulatory regions such as promoters or cis binding sites for regulatory factors, or by truncation of the coding sequence at any of a number of locations. These latter aberrations, resulting in less than total deletion of the gene set can be useful, for example, when rapid assessments of the coupling of a product are desired or when genetic reversion is less likely to occur.

To identify additional productive solutions to the above described bilevel OptKnock problem which lead to further sets of reactions to disrupt or metabolic modifications that can result in the biosynthesis, including growth-coupled biosynthesis of a desired product, an optimization method, termed integer cuts, can be implemented. This method proceeds by iteratively solving the OptKnock problem exemplified above with the incorporation of an additional constraint referred to as an integer cut at each iteration. Integer cut constraints effectively prevent the solution procedure from choosing the exact same set of reactions identified in any previous iteration that obligatorily couples product biosynthesis to growth. For example, if a previously identified growth-coupled metabolic modification specifies reactions 1, 2, and 3 for disruption, then the following constraint prevents the same reactions from being simultaneously considered in subsequent solutions. The integer cut method is well known in the art and can be found described in, for example, Burgard et al., Biotechnol. Prog. 17:791-797 (2001). As with all methods described herein with reference to their use in combination with the OptKnock computational framework for metabolic modeling and simulation, the integer cut method of reducing redundancy in iterative computational analysis also can be applied with other computational frameworks well known in the art including, for example, SimPheny®.

The methods exemplified herein allow the construction of cells and organisms that biosynthetically produce a desired product, including the obligatory coupling of production of a target biochemical product to growth of the cell or organism engineered to harbor the identified genetic alterations. Therefore, the computational methods described herein allow the identification and implementation of metabolic modifications that are identified by an in silico method selected from OptKnock or SimPheny®. The set of metabolic modifications can include, for example, addition of one or more biosynthetic pathway enzymes and/or functional disruption of one or more metabolic reactions including, for example, disruption by gene deletion.

As discussed above, the OptKnock methodology was developed on the premise that mutant microbial networks can be evolved towards their computationally predicted maximum-growth phenotypes when subjected to long periods of growth selection. In other words, the approach leverages an organism's ability to self-optimize under selective pressures. The OptKnock framework allows for the exhaustive enumeration of gene deletion combinations that force a coupling between biochemical production and cell growth based on network stoichiometry. The identification of optimal gene/reaction knockouts requires the solution of a bilevel optimization problem that chooses the set of active reactions such that an optimal growth solution for the resulting network overproduces the biochemical of interest (Burgard et al., Biotechnol. Bioeng. 84:647-657 (2003)).

An in silico stoichiometric model of E. coli metabolism can be employed to identify essential genes for metabolic pathways as exemplified previously and described in, for example, U.S. patent publications US 2002/0012939, US 2003/0224363, US 2004/0029149, US 2004/0072723, US 2003/0059792, US 2002/0168654 and US 2004/0009466, and in U.S. Pat. No. 7,127,379. As disclosed herein, the OptKnock mathematical framework can be applied to pinpoint gene deletions leading to the growth-coupled production of a desired product. Further, the solution of the bilevel OptKnock problem provides only one set of deletions. To enumerate all meaningful solutions, that is, all sets of knockouts leading to growth-coupled production formation, an optimization technique, termed integer cuts, can be implemented. This entails iteratively solving the OptKnock problem with the incorporation of an additional constraint referred to as an integer cut at each iteration, as discussed above.

As disclosed herein, a nucleic acid encoding a desired activity of a 1,4-cyclohexanedimethanol pathway can be introduced into a host organism. In some cases, it can be desirable to modify an activity of a 1,4-cyclohexanedimethanol pathway enzyme or protein to increase production of 1,4-cyclohexanedimethanol. For example, known mutations that increase the activity of a protein or enzyme can be introduced into an encoding nucleic acid molecule. Additionally, optimization methods can be applied to increase the activity of an enzyme or protein and/or decrease an inhibitory activity, for example, decrease the activity of a negative regulator.

One such optimization method is directed evolution. Directed evolution is a powerful approach that involves the introduction of mutations targeted to a specific gene in order to improve and/or alter the properties of an enzyme. Improved and/or altered enzymes can be identified through the development and implementation of sensitive high-throughput screening assays that allow the automated screening of many enzyme variants (for example, >10⁴). Iterative rounds of mutagenesis and screening typically are performed to afford an enzyme with optimized properties. Computational algorithms that can help to identify areas of the gene for mutagenesis also have been developed and can significantly reduce the number of enzyme variants that need to be generated and screened. Numerous directed evolution technologies have been developed (for reviews, see Hibbert et al., Biomol. Eng 22:11-19 (2005); Huisman and Lalonde, In Biocatalysis in the pharmaceutical and biotechnology industries pgs. 717-742 (2007), Patel (ed.), CRC Press; Otten and Quax. Biomol. Eng 22:1-9 (2005); and Sen et al., Appl Biochem. Biotechnol 143:212-223 (2007)) to be effective at creating diverse variant libraries, and these methods have been successfully applied to the improvement of a wide range of properties across many enzyme classes. Enzyme characteristics that have been improved and/or altered by directed evolution technologies include, for example: selectivity/specificity, for conversion of non-natural substrates; temperature stability, for robust high temperature processing; pH stability for bioprocessing under lower or higher pH conditions; substrate or product tolerance so that high product titers can be achieved; binding (Kₘ), including broadening substrate binding to include non-natural substrates; inhibition (Kᵢ), to remove inhibition by products, substrates, or key intermediates; activity (kcat), to increases enzymatic reaction rates to achieve desired flux; expression levels, to increase protein yields and overall pathway flux; oxygen stability, for operation of air sensitive enzymes under aerobic conditions; and anaerobic activity, for operation of an aerobic enzyme in the absence of oxygen.

A number of exemplary methods have been developed for the mutagenesis and diversification of genes to target desired properties of specific enzymes. Such methods are well known to those skilled in the art. Any of these can be used to alter and/or optimize the activity of a 1,4-cyclohexanedimethanol pathway enzyme or protein. Such methods include, but are not limited to EpPCR, which introduces random point mutations by reducing the fidelity of DNA polymerase in PCR reactions (Pritchard et al., J Theor. Biol. 234:497-509 (2005)); Error-prone Rolling Circle Amplification (epRCA), which is similar to epPCR except a whole circular plasmid is used as the template and random 6-mers with exonuclease resistant thiophosphate linkages on the last 2 nucleotides are used to amplify the plasmid followed by transformation into cells in which the plasmid is re-circularized at tandem repeats (Fujii et al., Nucleic Acids Res. 32:e145 (2004); and Fujii et al., Nat. Protoc. 1:2493-2497 (2006)); DNA or Family Shuffling, which typically involves digestion of two or more variant genes with nucleases such as Dnase I or EndoV to generate a pool of random fragments that are reassembled by cycles of annealing and extension in the presence of DNA polymerase to create a library of chimeric genes (Stemmer, Proc Natl Acad Sci USA 91:10747-10751 (1994); and Stemmer, Nature 370:389-391 (1994)); Staggered Extension (StEP), which entails template priming followed by repeated cycles of 2 step PCR with denaturation and very short duration of annealing/extension (as short as 5 sec) (Zhao et al., Nat. Biotechnol. 16:258-261 (1998)); Random Priming Recombination (RPR), in which random sequence primers are used to generate many short DNA fragments complementary to different segments of the template (Shao et al., Nucleic Acids Res 26:681-683 (1998)).

Additional methods include Heteroduplex Recombination, in which linearized plasmid DNA is used to form heteroduplexes that are repaired by mismatch repair (Volkov et al, Nucleic Acids Res. 27:e18 (1999); and Volkov et al., Methods Enzymol. 328:456-463 (2000)); Random Chimeragenesis on Transient Templates (RACHITT), which employs Dnase I fragmentation and size fractionation of single stranded DNA (ssDNA) (Coco et al., Nat. Biotechnol. 19:354-359(2001)); Recombined Extension on Truncated templates (RETT), which entails template switching of unidirectionally growing strands from primers in the presence of unidirectional ssDNA fragments used as a pool of templates (Lee et al., J. Molec. Catalysis 26:119-129 (2003)); Degenerate Oligonucleotide Gene Shuffling (DOGS), in which degenerate primers are used to control recombination between molecules; (Bergquist and Gibbs, Methods Mol. Biol 352:191-204 (2007); Bergquist et al., Biomol. Eng 22:63-72 (2005); Gibbs et al., Gene 271:13-20 (2001)); Incremental Truncation for the Creation of Hybrid Enzymes (ITCHY), which creates a combinatorial library with 1 base pair deletions of a gene or gene fragment of interest (Ostermeier et al., Proc. Natl. Acad. Sci. USA 96:3562-3567 (1999); and Ostermeier et al., Nat. Biotechnol.)7:1205-1209 (1999)); Thio-Incremental Truncation for the Creation of Hybrid Enzymes (THIO-ITCHY), which is similar to ITCHY except that phosphothioate dNTPs are used to generate truncations (Lutz et al., Nucleic Acids Res 29:E16 (2001)); SCRATCHY, which combines two methods for recombining genes, ITCHY and DNA shuffling (Lutz et al., Proc. Natl. Acad Sci. USA 98:11248-11253 (2001)); Random Drift Mutagenesis (RNDM), in which mutations made via epPCR are followed by screening/selection for those retaining usable activity (Bergquist et al., Biomol. Eng. 22:63-72 (2005)); Sequence Saturation Mutagenesis (SeSaM), a random mutagenesis method that generates a pool of random length fragments using random incorporation of a phosphothioate nucleotide and cleavage, which is used as a template to extend in the presence of “universal” bases such as inosine, and replication of an inosine-containing complement gives random base incorporation and, consequently, mutagenesis (Wong et al., Biotechnol. J. 3:74-82 (2008); Wong et al., Nucleic Acids Res. 32:e26 (2004); and Wong et al., Anal. Biochem. 341:187-189 (2005)); Synthetic Shuffling, which uses overlapping oligonucleotides designed to encode “all genetic diversity in targets” and allows a very high diversity for the shuffled progeny (Ness et al., Nat. Biotechnol. 20:1251-1255 (2002)); Nucleotide Exchange and Excision Technology NexT, which exploits a combination of dUTP incorporation followed by treatment with uracil DNA glycosylase and then piperidine to perform endpoint DNA fragmentation (Muller et al., Nucleic Acids Res. 33:e117 (2005)).

Further methods include Sequence Homology-Independent Protein Recombination (SHIPREC), in which a linker is used to facilitate fusion between two distantly related or unrelated genes, and a range of chimeras is generated between the two genes, resulting in libraries of single-crossover hybrids (Sieber et al., Nat. Biotechnol. 19:456-460 (2001)); Gene Site Saturation Mutagenesis™ (GSSM™), in which the starting materials include a supercoiled double stranded DNA (dsDNA) plasmid containing an insert and two primers which are degenerate at the desired site of mutations (Kretz et al., Methods Enzymol. 388:3-11 (2004)); Combinatorial Cassette Mutagenesis (CCM), which involves the use of short oligonucleotide cassettes to replace limited regions with a large number of possible amino acid sequence alterations (Reidhaar-Olson et al. Methods Enzymol. 208:564-586 (1991); and Reidhaar-Olson et al. Science 241:53-57 (1988)); Combinatorial Multiple Cassette Mutagenesis (CMCM), which is essentially similar to CCM and uses epPCR at high mutation rate to identify hot spots and hot regions and then extension by CMCM to cover a defined region of protein sequence space (Reetz et al., Angew. Chem. Int. Ed Engl. 40:3589-3591 (2001)); the Mutator Strains technique, in which conditional is mutator plasmids, utilizing the mutD5 gene, which encodes a mutant subunit of DNA polymerase III, to allow increases of 20 to 4000-X in random and natural mutation frequency during selection and block accumulation of deleterious mutations when selection is not required (Selifonova et al., Appl. Environ. Microbiol. 67:3645-3649 (2001)); Low et al., J. Mol. Biol. 260:359-3680 (1996)).

Additional exemplary methods include Look-Through Mutagenesis (LTM), which is a multidimensional mutagenesis method that assesses and optimizes combinatorial mutations of selected amino acids (Rajpal et al., Proc. Natl. Acad. Sci. USA 102:8466-8471 (2005)); Gene Reassembly, which is a DNA shuffling method that can be applied to multiple genes at one time or to create a large library of chimeras (multiple mutations) of a single gene (Tunable GeneReassembly™ (TGR™) Technology supplied by Verenium Corporation), in Silico Protein Design Automation (PDA), which is an optimization algorithm that anchors the structurally defined protein backbone possessing a particular fold, and searches sequence space for amino acid substitutions that can stabilize the fold and overall protein energetics, and generally works most effectively on proteins with known three-dimensional structures (Hayes et al., Proc. Natl. Acad. Sci. USA 99:15926-15931 (2002)); and Iterative Saturation Mutagenesis (ISM), which involves using knowledge of structure/function to choose a likely site for enzyme improvement, performing saturation mutagenesis at chosen site using a mutagenesis method such as Stratagene QuikChange (Stratagene; San Diego Calif.), screening/selecting for desired properties, and, using improved clone(s), starting over at another site and continue repeating until a desired activity is achieved (Reetz et al., Nat. Protoc. 2:891-903 (2007); and Reetz et al., Angew. Chem. Int. Ed Engl. 45:7745-7751 (2006)).

Any of the aforementioned methods for mutagenesis can be used alone or in any combination. Additionally, any one or combination of the directed evolution methods can be used in conjunction with adaptive evolution techniques, as described herein.

It is understood that modifications which do not substantially affect the activity of the various embodiments of this invention are also included within the definition of the invention provided herein. Accordingly, the following examples are intended to illustrate but not limit the present invention.

### Example I

### Organisms for the Production of 1,4-Cyclohexanedimethanol

This Example shows enzymes useful in the construction of an organism that can be used in the production of 1,4-cyclohexanedimethanol.

Table 1 below summarizes the enzyme classes for the enzymes shown in pathways of FIG. 1.

TABLE 1

| Label | Function | Step |
|---|---|---|
| 1.1.1.a | Oxidoreductase (aldehyde to alcohol) | 1C, H |
| 1.2.1.b | Oxidoreductase (acyl-CoA to aldehyde) | 1I, M |
| 1.2.1.d | Oxidoreductase (phosphorylating/dephosphorylating) | 1G, P |
| 1.2.1.e | Oxidoreductase (acid to aldehyde) | 1B, J |
| 1.14. | Monooxygenases | 1A,K |
| 2.3.1.a | Acyltransferase (transferring phosphate group to CoA; | 1F, O |
| | phosphotransacylase) | |
| 2.7.2.a | Phosphotransferase, carboxyl group acceptor (kinase) | 1E, N |
| 2.8.3.a | Coenzyme-A transferase | 1D, L |
| 3.1.2.a | Thiolester hydrolase (CoA specific) | 1D, L |
| 6.2.1.a | Acid-thiol ligase | 1D, L |

The reduction of p-hydroxymethyl benzaldehyde to dihydroxymethyl benzene and of p-methyl benzaldehyde to p-methylbenzyl alcohol (Steps C and H respectively in FIG. 1) can be catalyzed by oxidoreductases that transform the acetaldehyde group to the alcohol group. Aryl alcohol dehydrogenase (EC 1.1.1.90 and 1.1.1.91) can in general be used for converting an aromatic aldehyde into an alcohol. One exemplary enzyme is benzyl alcohol dehydrogenase that catalyzes the reversible conversion of benzyl alcohol to benzaldehyde (Shaw, J. P., et al., Eur J Biochem. 191:705-714 (1992). Specifically, the benzyl alcohol dehydrogenase from Solanam tuberosum has been shown to have activity on both p-methylbenzaldehyde and p-hydroxybenzaldehyde (Davies, D. D., et al., Phytochemistry 12:531-536 (1973)) among several other aromatic substrates but its gene has not been identified as yet. This enzyme from Pseudomonas putida has also been shown to act on other substrates such as 3-methylbenzyl alcohol, 3-chlorobenzyl alcohol and 4-methylbenzyl alcohol (Worsey, M. J., et al., J Bacteriol. 124:7-13 (1975)). Benzyl alcohol dehydrogenase from Acinetobacter calcoaceticus has also been shown to catalyze multiple aromatic substrates. The enzymes from Pseudomonas putida and A. calcoaceticus have been expressed in E. coli and were found to be effective catalysts for benzyl alcohol and 4-aminobenzyl alcohol (Curtis, A. J., et al., Biochem. Biophys Res. Commun. 259:220-223 (1999). Both these enzymes have been kinetically characterized. Benzyl alcohol dehydrogenase from a denitrifying Thauera sp. Has been purified (Beigert, T. A. U. E. C. F. G., Arch. Microbiol 163:418-423 (1995)) and has been shown to act on benzyl alcohol and p-hydroxy benzyl alcohol. Yet another gene candidate for this step is 4-carboxybenzyl alcohol dehydrogenase that interconverts 4-Carboxybenzyl alcohol and its corresponding aldehyde. The aryl alcohol dehydrogenase from the white-rot fungus, Phanerochaete chrysosporium has been characterized and has been shown to function on multiple substrates, including 3,5-dimethoxybenzaldehyde, 4-methoxybenzaldehyde, phenylacetaldehyde, benzaldehyde, p-hydroxybenzaldehyde, and 3,4-dihydroxybenzaldehyde (Muheim, A., et al., Eur J Biochem. 195:369-375 (1991)). This enzyme has been cloned and expressed in E. coli (Reiser, J., et al., J Biol Chem 269:28152-28159 (1994)). This enzyme has been characterized from other white rot fungi also (Gutierrez, A., et al., Appl Environ Microbiol 60:1783-1788 (1994)), including Pluerotus eryngii (Guillen, F. et al., Appl Environ Microbiol 60:2811-2817 (1994)).

Alcohol dehydrogenases from Saccharomyces cerevisiae that reduce 4-hydroxyphenyl acetaldehyde to tyrosol in the tyrosine degradation pathway are other candidates for catalyzing this transformation. This reaction can be catalyzed by ADH-I, II, III, IV and V in S. cerevisiae. Any one of these alcohol dehydrogenases can also catalyze the reduction of 2-phenylacetaldehyde to 2-phenylethanol and of 2-indoleacetaldehyde to tryptophol. Formaldehyde dehydrogenase in yeast encoded by Sfa1p was also demonstrated to have the ability to reduce long chain complex aldehydes to alcohols (Dickinson, J. R., et al., J Biol Chem 278:8028-8034 (2003)).

| Gene | GenBank ID | GI number | Organism |
|---|---|---|---|
| xylB | YP_003617171 | 296100254 | Pseudomonas putida mt-2 |
| Adh1 | NP_014555.1 | 6324486 | Saccharomyces cerevisiae |
| Adh2 | NP_014032.1 | 6323961 | Saccharomyces cerevisiae |
| Adh3 | NP_013800.1 | 6323729 | Saccharomyces cerevisiae |
| Adh4 | NP_011258.2 | 269970305 | Saccharomyces cerevisiae |
| Adh5 | NP_009703.1 | 6319621 | Saccharomyces cerevisiae |
| tsaC | AAC44807.1 | 1790870 | Comamonas testosterone |
| aad | Q01752.1 | 2492798 | Phanerochaete chrysosporium |
| xylB | AAC32671.1 | 1408294 | Acinetobacter calcoaceticus |

Other exemplary genes encoding enzymes that catalyze the conversion of an aldehyde to alcohol (i.e., alcohol dehydrogenase or equivalently aldehyde reductase) include alrA encoding a medium-chain alcohol dehydrogenase for C2-C14 (Tani, A., et al., Appl. Environ. Microbiol. 66:5231-5235 (2000)), yqhD from E. coli which has preference for molecules longer than C(3) (Sulzenbacher, G., et al., Journal of Molecular Biology 342:489-502 (2004)), and bdh I and bdh II from C. acetobutylicum which converts butyryaldehyde into butanol (Walter, K. A., Journal of Bacteriology 174:7149-7158 (1993)). The gene product of yqhD catalyzes the reduction of acetaldehyde, malondialdehyde, propionaldehyde, butyraldehyde, and acrolein using NADPH as the cofactor (Perez, J. M., et al., J Biol. Chem. 283:7346-7353 (2008)). (Perez, J. M., et al., J Biol. Chem. 283:7346-7353 (2008)). The adhA gene product from Zymomonas mobilis has been demonstrated to have activity on a number of aldehydes including formaldehyde, acetaldehyde, propionaldehyde, butyraldehyde, and acrolein (Kinoshita, S., et al., Appl Microbiol Biotechnol 22:249-254 (1985)). Additional aldehyde reductase candidates are encoded by bdh in C. saccharoperbutylacetonicum and Cbei_{—}1722, Cbei_{—}2181 and Cbei2421 in C. beijerinckii.

| Gene | GenBank ID | GI number | Organism |
|---|---|---|---|
| alrA | BAB12273.1 | 9967138 | Acinetobacter sp. strain |
| | | | M-1 |
| yqhD | NP_417484.1 | 16130909 | Escherichia coli |
| bdh I | NP_349892.1 | 15896543 | Clostridium |
| | | | acetobutylicum |
| bdh II | NP_349891.1 | 15896542 | Clostridium |
| | | | acetobutylicum |
| adhA | YP_162971.1 | 56552132 | Zymomonas mobilis |
| bdh | BAF45463.1 | 124221917 | Clostridium |
| | | | saccharoperbutyl- |
| | | | acetonicum |
| Cbei_1722 | YP_001308850 | 150016596 | Clostridium beijerinckii |
| Cbei_2181 | YP_001309304 | 150017050 | Clostridium beijerinckii |
| Cbei_2421 | YP_001309535 | 150017281 | Clostridium beijerinckii |

Enzymes exhibiting 4-hydroxybutyrate dehydrogenase activity (EC 1.1.1.61) also fall into this category. Such enzymes have been characterized in Ralstonia eutropha (Bravo, D. T., et al., J. Forensic Sci. 49:379-387 (2004)), Clostridium kluyveri (Wolff, R. A., et al., Protein Expr. Purif. 6:206-212 (1995)) and Arabidopsis thaliana (Breitkreuz, K. E., et al., J. Biol. Chem. 278:41552-41556 (2003)). The A. thaliana enzyme was cloned and characterized in yeast (Breitkreuz, K. E., et al., J. Biol. Chem. 278:41552-41556 (2003)). Yet another gene is the alcohol dehydrogenase adhI from Geobacillus thermoglucosidasius (Jeon, Y. J., et al., J Biotechnol 135:127-133 (2008)).

| Gene | GenBank ID | GI number | Organism |
|---|---|---|---|
| hbd | YP_726053.1 | 113867564 | Ralstonia eutropha H16 |
| hbd | L21902.1 | 146348486 | Clostridium kluyveri DSM 555 |
| hbd | Q94B07 | 75249805 | Arabidopsis thaliana |
| dhI | AAR91477.1 | 40795502 | Geobacillus thermoglucosidasius |

Another exemplary enzyme is 3-hydroxyisobutyrate dehydrogenase (EC 1.1.1.31) which catalyzes the reversible oxidation of 3-hydroxyisobutyrate to methylmalonate semialdehyde. This enzyme participates in valine, leucine and isoleucine degradation and has been identified in bacteria, eukaryotes, and mammals. The enzyme encoded by P84067 from Thermus thermophilus HB8 has been structurally characterized (Lokanath, N. K., et al., J Mol Biol 352:905-17 (2005)). The reversibility of the human 3-hydroxyisobutyrate dehydrogenase was demonstrated using isotopically-labeled substrate (Manning, N. J. et al., Biochem J 231:481-4 (1985)). Additional genes encoding this enzyme include 3hidh in Homo sapiens (Hawes, J. W., et al., Methods Enzymol. 324:218-228 (2007) and Oryctolagus cuniculus (Chowdhury, E. K., et al., Biosci. Biotechnol Biochem. 60:2043-2047 (1996). (Hawes, J. W., et al., Methods Enzymol. 324:218-228 (2007)), mmsB in Pseudomonas aeruginosa and Pseudomonas putida, and dhat in Pseudomonas putida (Aberhart, D. J., J Chem. Soc. [Perkin 1] 6:1404-1406 (1979)), (Chowdhury, E. K., et al., Biosci. Biotechnol Biochem. 67:438-441 (2003)). (Chowdhury, E. K., et al., Biosci. Biotechnol Biochem. 60:2043-2047 (2003)). Several 3-hydroxyisobutyrate dehydrogenase enzymes have been characterized in the reductive direction, including mmsB from Pseudomonas aeruginosa (Gokarn, R. R., et al., U.S. Pat. No. 7,393,676 (2008)) and mmsB from Pseudomonas putida.

| Gene | GenBank ID | GI number | Organism |
|---|---|---|---|
| P84067 | P84067 | 75345323 | Thermus thermophilus |
| 3hidh | P31937.2 | 12643395 | Homo sapiens |
| 3hidh | P32185.1 | 416872 | Oryctolagus cuniculus |
| mmsB | NP_746775.1 | 26991350 | Pseudomonas putida |
| mmsB | P28811.1 | 127211 | Pseudomonas aeruginosa |
| dhat | Q59477.1 | 2842618 | Pseudomonas putida |

3-Hydroxypropionate dehydrogenase, also known as malonate semialdehyde reductase, catalyzes the reversible conversion of malonic semialdehyde to 3-HP. An NADH-dependent 3-hydroxypropionate dehydrogenase is thought to participate in beta-alanine biosynthesis pathways from propionate in bacteria and plants (Rathinasabapathi B., Journal of Plant Pathology 159:671-674 (2002)). (Stadtman, E. R., A. J. Am. Chem. Soc. 77:5765-5766 (1955)). This enzyme has not been associated with a gene in any organism to date. An NADPH-dependent malonate semialdehyde reductase catalyzes the reverse reaction in autotrophic CO₂-fixing bacteria. Although the enzyme activity has been detected in Metallosphaera sedula, the identity of the gene is not known (Alber, B., J. Bacteriol. 188:8551-8559 (2006)). Several 3-hydroxyisobutyrate dehydrogenase enzymes exhibit 3-hydroxypropionate dehydrogenase activity. Three genes exhibiting this activity are mmsB from Pseudomonas aeruginosa PAO1 (Gokarn, R. R., et al., U.S. Pat. No. 7,393,676 (2008)), mmsB from Pseudomonas putida KT2440 and mmsB from Pseudomonas putida E23 (Chowdhury, E. K., et al., Biosci. Biotechnol. Biochem. 60:2043-2047 (1996)).

| GENE | GENBANK ID | GI NUMBER | ORGANISM |
|---|---|---|---|
| mmsB | NP_252259.1 | 15598765 | Pseudomonas putida |
| mmsB | NP_746775.1 | 26991350 | Pseudomonas aeruginosa |
| mmsB | JC7926 | 60729613 | Pseudomonas putida |

The reduction of p-methylbenzoyl-CoA to p-methylbenzaldehyde (also called p-tolualdehyde) is catalyzed by an enzyme with p-methylbenzoyl-CoA reductase activity (Step I of FIG. 1). Similarly, p-hydroxymethyl benzoyl-CoA reductase catalyzes the reduction of p-hydroxymethyl benzoyl-CoA to p-hydroxymethyl benzaldehyde (Step M of FIG. 1). Enzymes with these activities have not been characterized to date. An enzyme catalyzing a similar reaction is cinnamoyl-CoA reductase (EC 1.2.1.44). This enzyme catalyzes the NAD(P)H-dependent reduction of cinnamoyl-CoA and substituted aromatic derivatives such as coumaroyl-CoA and feruloyl-CoA. The enzyme has been characterized in organisms including Arabidopsis thaliana (Lauvergeat et al., Phytochemistry 57:1187-1195 (2001)), Triticum aestivum (Ma, J Exp. Bot. 58:2011-2021 (2007)) and Panicum virgatum (Escamilla-Trevino et al., New Phytol. 185:143-155 (2010)). The enzymes from A. thaliana and P. virgatum were characterized and heterologously expressed in E. coli.

| | GenBank Accession | | |
|---|---|---|---|
| Gene | No. | GI No. | Organism |
| TACCR1 | ABE01883.1 | 90902167 | Triticum aestivum |
| AtCCR1 | AAU45042.1 | 52355804 | Arabidopsis thaliana |
| AtCCR2 | AAG53687.1 | 12407990 | Arabidopsis thaliana |
| PvCCR1 | ACZ74580.1 | 270315096 | Panicum virgatum |
| PvCCR2 | ACZ74585.1 | 270315106 | Panicum virgatum |

Several other well-characterized acyl-CoA reductases reduce an acyl-CoA to its corresponding aldehyde. Exemplary enzymes include fatty acyl-CoA reductase (EC 1.2.1.50), succinyl-CoA reductase (EC 1.2.1.76), acetyl-CoA reductase (EC 1.2.1.10) and butyryl-CoA reductase. Exemplary fatty acyl-CoA reductases enzymes are encoded by acr1 of Acinetobacter calcoaceticus (Reiser et al., 179:2969-2975 (1997)) and Acinetobacter sp. M-1 (Ishige et al., Appl. Environ. Microbiol. 68:1192-1195 (2002)). Enzymes with succinyl-CoA reductase activity are encoded by sucD of Clostridium kluyveri (Sohling et al., J Bacteriol. 178:871-880 (1996a); Sohling et al., 178:871-80 (1996)) and sucD of P. gingivalis (Takahashi et al., J. Bacteriol. 182:4704-4710 (2000)). Additional succinyl-CoA reductase enzymes participate in the 3-hydroxypropionate/4-hydroxybutyrate cycle of thermophilic archaea such as Metallosphaera sedula (Berg et al., Science. 318:1782-)786 (2007)) and Thermoproteus neutrophilus (Ramos-Vera et al., J Bacteriol. 191:4286-4297 (2009)). The M. sedula CoA reductase, encoded by Msed_{—}0709, is strictly NADPH-dependent and also has malonyl-CoA reductase activity. The T. neutrophilus enzyme is active with both NADPH and NADH. The acylating acetaldehyde dehydrogenase in Pseudomonas sp, encoded by bphG, has been demonstrated to oxidize and acylate acetaldehyde, propionaldehyde, butyraldehyde, isobutyraldehyde and formaldehyde to their corresponding CoA-esters (Powlowski et al., 175:377-385 (1993)). In addition to reducing acetyl-CoA to ethanol, the enzyme encoded by adhE in Leuconostoc mesenteroides has been shown to oxidize the branched chain compound isobutyraldehyde to isobutyryl-CoA (Koo et al., Biotechnol Lett. 27:505-510 (2005)). Butyraldehyde dehydrogenase catalyzes a similar reaction, conversion of butyryl-CoA to butyraldehyde, in solventogenic organisms such as Clostridium saccharoperbutylacetonicum (Kosaka et al., Biosci. Biotechnol Biochem. 71:58-68 (2007)). The acyl-CoA reductase encoded by ald in Clostridium beijerinckii has been reported to reduce acetyl-CoA and butyryl-CoA to their corresponding aldehydes (Toth et al., Appl Environ. Microbiol 65:4973-4980 (1999)). This enzyme exhibits high sequence homology to the CoA-dependent acetaldehyde dehydrogenase enzymes of Salmonella typhimurium and E. coli encoded by eutE (Toth et al., Appl Environ. Microbiol 65:4973-4980 (1999)).

| Gene | GenBank Accession No. | GI No. | Organism |
|---|---|---|---|
| acr1 | YP_047869.1 | 50086359 | Acinetobacter calcoaceticus |
| acr1 | AAC45217 | 1684886 | Acinetobacter baylyi |
| acr1 | BAB85476.1 | 18857901 | Acinetobacter sp. Strain M-1 |
| MSED_0709 | YP_001190808.1 | 146303492 | Metallosphaera sedula |
| Tneu_0421 | ACB39369.1 | 170934108 | Thermoproteus neutrophilus |
| sucD | P38947.1 | 172046062 | Clostridium kluyveri |
| sucD | NP_904963.1 | 34540484 | Porphyromonas gingivalis |
| bphG | BAA03892.1 | 425213 | Pseudomonas sp |
| adhE | AAV66076.1 | 55818563 | Leuconostoc mesenteroides |
| bld | AAP42563.1 | 31075383 | Clostridium |
| | | | saccharoperbutylacetonicum |
| ald | AAT66436 | 9473535 | Clostridium beijerinckii |
| eutE | AAA80209 | 687645 | Salmonella typhimurium |
| eutE | P77445 | 2498347 | Escherichia coli |

An additional CoA reductase enzyme candidate is malonyl-CoA reductase which transforms malonyl-CoA to malonic semialdehyde. Malonyl-CoA reductase is a key enzyme in autotrophic carbon fixation via the 3-hydroxypropionate cycle in thermoacidophilic archael bacteria (Berg et al., 318:1782-1786 (2007); Thauer, 318:1732-1733 (2007)). The enzyme utilizes NADPH as a cofactor and has been characterized in Metallosphaera and Sulfolobus spp (Alber et al., 188:8551-8559 (2006); Hugler et al., 184:2404-2410 (2002)). The enzyme is encoded by Msed 0709 in Metallosphaera sedula (Alber et al., 188:8551-8559 (2006); Berg et al., 318:1782-1786 (2007)). A gene encoding a malonyl-CoA reductase from Sulfolobus tokodaii was cloned and heterologously expressed in E. coli (Alber et al., 188:8551-8559 (2006)). This enzyme has also been shown to catalyze the conversion of methylmalonyl-CoA to its corresponding aldehyde (WO/2007/141208). Both malonyl-CoA reductase enzyme candidates have high sequence similarity to aspartate-semialdehyde dehydrogenase, an enzyme catalyzing the reduction and concurrent dephosphorylation of aspartyl-4-phosphate to aspartate semialdehyde. Additional gene candidates can be found by sequence homology to proteins in other organisms including Sulfolobus solfataricus and Sulfolobus acidocaldarius.

| | GenBank | | |
|---|---|---|---|
| | Accession | | |
| Gene | No. | GI No. | Organism |
| MSED_0709 | YP_001190808.1 | 146303492 | Metallosphaera sedula |
| mcr | NP_378167.1 | 15922498 | Sulfolobus tokodaii |
| asd-2 | NP_343563.1 | 15898958 | Sulfolobus solfataricus |
| Saci_2370 | YP_256941.1 | 70608071 | Sulfolobus |
| | | | acidocaldarius |

The reduction of p-methylbenzoyloxy-phosphonate to p-methylbenzaldehyde (Step G of FIG. 1) and of p-hydroxymethylbenzoyloxy-phosphonate to p-hydroxymethyl benzaldehyde (Step P of FIG. 1) is catalyzed by enzymes with phosphonate reductase activities. Although enzymes catalyzing these conversions have not been identified to date, similar transformations catalyzed by glyceraldehyde-3-phosphate dehydrogenase (EC 1.2.1.12), aspartate-semialdehyde dehydrogenase (EC 1.2.1.11) acetylglutamylphosphate reductase (EC 1.2.1.38) and glutamate-5-semialdehyde dehydrogenase (EC 1.2.1.) are well documented. Aspartate semialdehyde dehydrogenase (ASD, EC 1.2.1.11) catalyzes the NADPH-dependent reduction of 4-aspartyl phosphate to aspartate-4-semialdehyde. ASD participates in amino acid biosynthesis and recently has been studied as an antimicrobial target (Hadfield et al., Biochemistry 40:14475-14483 (2001)). The E. coli ASD structure has been solved (Hadfield et al., J Mol. Biol. 289:991-1002 (1999)) and the enzyme has been shown to accept the alternate substrate beta-3-methylaspartyl phosphate (Shames et al., J Biol. Chem. 259:15331-15339 (1984)). The Haemophilus influenzae enzyme has been the subject of enzyme engineering studies to alter substrate binding affinities at the active site (Blanco et al., Acta Crystallogr. D. Biol. Crystallogr. 60:1388-1395 (2004)). Other ASD candidates are found in Mycobacterium tuberculosis (Shafiani et al., J Appl Microbiol 98:832-838 (2005)), Methanococcus jannaschii (Faehnle et al., J Mol. Biol. 353:1055-1068 (2005)), and the infectious microorganisms Vibrio cholera and Helicobacter pylori (Moore et al., Protein Expr. Purif 25:189-194 (2002)). A related enzyme candidate is acetylglutamylphosphate reductase (EC 1.2.1.38), an enzyme that naturally reduces acetylglutamylphosphate to acetylglutamate-5-semialdehyde, found in S. cerevisiae (Pauwels et al., Eur. J Biochem. 270:1014-1024 (2003)), B. subtilis (O'Reilly et al., Microbiology 140 (Pt 5):1023-1025 (1994)), E. coli (Parsot et al., Gene. 68:275-283 (1988)), and other organisms. Additional phosphate reductase enzymes of E. coli include glyceraldehyde 3-phosphate dehydrogenase encoded by gapA (Branlant et al., Eur. J. Biochem. 150:61-66 (1985)) and glutamate-5-semialdehyde dehydrogenase encoded by proA (Smith et al., J. Bacteriol. 157:545-551 (1984b)). Genes encoding glutamate-5-semialdehyde dehydrogenase enzymes from Salmonella typhimurium (Mahan et al., J Bacteriol. 156:1249-1262 (1983)) and Campylobacter jejuni (Louie et al., Mol. Gen. Genet. 240:29-35 (1993)) were cloned and expressed in E. coli.

| Gene | GenBank ID | GI Number | Organism |
|---|---|---|---|
| asd | NP_417891.1 | 16131307 | Escherichia coli |
| asd | YP_248335.1 | 68249223 | Haemophilus influenzae |
| asd | AAB49996 | 1899206 | Mycobacterium tuberculosis |
| VC2036 | NP_231670 | 15642038 | Vibrio cholera |
| asd | YP_002301787.1 | 210135348 | Helicobacter pylori |
| ARG5,6 | NP_010992.1 | 6320913 | Saccharomyces cerevisiae |
| argC | NP_389001.1 | 16078184 | Bacillus subtilis |
| argC | NP_418393.1 | 16131796 | Escherichia coli |
| gapA | P0A9B2.2 | 71159358 | Escherichia coli |
| proA | NP_414778.1 | 16128229 | Escherichia coli |
| proA | NP_459319.1 | 16763704 | Salmonella typhimurium |
| proA | P53000.2 | 9087222 | Campylobacter jejuni |

Direct conversion of p-hydroxymethyl benzoate to p-hydroxymethyl benzaldehyde and p-toluate to p-methylbenzaldehyde (Steps B and J of FIG. 1) is catalyzed by a carboxylic acid reductase. Exemplary enzymes include carboxylic acid reductase, alpha-aminoadipate reductase and retinoic acid reductase. Carboxylic acid reductase (CAR) catalyzes the magnesium, ATP and NADPH-dependent reduction of carboxylic acids to their corresponding aldehydes (Venkitasubramanian et al., J Biol. Chem. 282:478-485 (2007)). The natural substrate of this enzyme is benzoate and the enzyme exhibits broad acceptance of aromatic substrates including p-toluate (Venkitasubramanian et al., Biocatalysis in Pharmaceutical and Biotechnology Industries. CRC press (2006)). The enzyme from Nocardia iowensis, encoded by car, was cloned and functionally expressed in E. coli (Venkitasubramanian et al., J Biol. Chem. 282:478-485 (2007)). CAR requires post-translational activation by a phosphopantetheine transferase (PPTase) that converts the inactive apo-enzyme to the active holo-enzyme (Hansen et al., Appl. Environ. Microbiol 75:2765-2774 (2009)). Expression of the npt gene, encoding a specific PPTase, improved activity of the enzyme. A similar enzyme found in Streptomyces griseus is encoded by the griC and griD genes. This enzyme is believed to convert 3-amino-4-hydroxybenzoic acid to 3-amino-4-hydroxybenzaldehyde, as deletion of either griC or griD led to accumulation of extracellular 3-acetylamino-4-hydroxybenzoic acid, a shunt product of 3-amino-4-hydroxybenzoic acid metabolism (Suzuki, et al., J. Antibiot. 60(6):380-387 (2007)). The S. griseus PPTase is likely encoded by SGR 665, as predicted by sequence homology to the Nocardia iowensis npt gene.

| | GenBank Accession | | |
|---|---|---|---|
| Gene | No. | GI No. | Organism |
| car | AAR91681.1 | 40796035 | Nocardia iowensis |
| npt | ABI83656.1 | 114848891 | Nocardia iowensis |
| griC | YP_001825755.1 | 182438036 | Streptomyces griseus |
| griD | YP_001825756.1 | 182438037 | Streptomyces griseus |
| SGR_665 | YP_001822177.1 | 182434458 | Streptomyces griseus |

An enzyme with similar characteristics, alpha-aminoadipate reductase (AAR, EC 1.2.1.31), participates in lysine biosynthesis pathways in some fungal species. This enzyme naturally reduces alpha-aminoadipate to alpha-aminoadipate semialdehyde. The carboxyl group is first activated through the ATP-dependent formation of an adenylate that is then reduced by NAD(P)H to yield the aldehyde and AMP. Like CAR, this enzyme utilizes magnesium and requires activation by a PPTase. Enzyme candidates for AAR and its corresponding PPTase are found in Saccharomyces cerevisiae (Morris et al., Gene 98:141-145 (1991)), Candida albicans (Guo et al., Mol. Genet. Genomics 269:271-279 (2003)), and Schizosaccharomyces pombe (Ford et al., Curr. Genet. 28:131-137 (1995)). The AAR from S. pombe exhibited significant activity when expressed in E. coli (Guo et al., Yeast 21:1279-1288 (2004)). The AAR from Penicillium chrysogenum accepts S-carboxymethyl-L-cysteine as an alternate substrate, but did not react with adipate, L-glutamate or diaminopimelate (Hijarrubia et al., J Biol. Chem. 278:8250-8256 (2003)). The gene encoding the P. chrysogenum PPTase has not been identified to date and no high-confidence hits were identified by sequence comparison homology searching.

| | GenBank | | |
|---|---|---|---|
| Gene | Accession No. | GI No. | Organism |
| LYS2 | AAA34747.1 | 171867 | Saccharomyces cerevisiae |
| LYS5 | P50113.1 | 1708896 | Saccharomyces cerevisiae |
| LYS2 | AAC02241.1 | 2853226 | Candida albicans |
| LYS5 | AAO26020.1 | 28136195 | Candida albicans |
| Lys1p | P40976.3 | 13124791 | Schizosaccharomyces pombe |
| Lys7p | Q10474.1 | 1723561 | Schizosaccharomyces pombe |
| Lys2 | CAA74300.1 | 3282044 | Penicillium chrysogenum |

The conversion of p-toluate to p-hydroxymethyl benzoate and p-methyl benzyl alcohol to dihydroxymethyl benzene (Steps A and K, FIG. 1) can be catalyzed by monoxygenases. One exemplary enzyme is toluene monoxygenase from Pseudomonas putida TW3 that converts toluene to benzyl alcohol. This enzyme has also been shown to convert 4-nitrotoluene to 4-nitrobenzyl alcohol. This is encoded by two genes, ntnM, ntnA, that are part of the ntn operon (James, K. D., et al., J Bacteriol. 180:2043-2049 (1998)). Xylene monooxygenase is another candidate for this transformation. This enzyme is a heterodimer composed of two subunits, XylA and Xy1M. This enzyme has broad substrate specificity and acts on toluene, p- and m-xylene, and 3-chlorotoluene to convert these substrates into benzyl alcohol, 4-methylbenzyl alcohol, 3-methylbenzyl alcohol and 3-cholorobenzyl alcohol respectively (Buhler, B., et al., J Biol Chem 275:10085-10092 (2000)). (Shaw, J. P., et al., Eur J Biochem. 209:51-61) 1992)). Yet another candidate is p-toluate methyl-monooxygenase that oxidizes p-toluate to 4-carboxybenzyl alcohol in the presence of O₂. The Comamonas testosteroni enzyme (tsaBM), which also reacts with 4-toluene sulfonate as a substrate, has been purified and characterized (Locher, H. H., et al., J Bacteriol. 173:3741-3748 (1991)). P-cymene monoxoygenase from P. putida F1 has also been shown to have broad substrate specificity with preference for substrates that had an alkyl or heteroatom substituent at the para-position of toluene (Nishio, T., et al., Appl Microbiol Biotechnol 55:321-325 (2001)).

| | GenBank | | |
|---|---|---|---|
| Gene | Accession No. | GI No. | Organism |
| tsaB | AAC44805.1 | 1790868 | Comamonas testosteroni |
| tsaM | AAC44804.1 | 1790867 | Comamonas testosteroni |
| xylA | YP_003617172.1 | 296100255 | Pseudomonas putida |
| xylM | CAC86827.1 | 18077191 | Pseudomonas putida |
| ntnM | AAC38359.1 | 2833678 | Pseudomonas putida TW3 |
| ntnA | AAC38360.1 | 2833679 | Pseudomonas putida TW3 |
| cymAa | ADI95378.1 | 298682317 | Pseudomonas putida |
| cymAb | ADI95379.1 | 298682318 | Pseudomonas putida |

An enzyme with phosphotrans-p-methylbenzoylase activity interconverts p-methylbenzoyl-CoA and (p-methylbenzoyloxy)phosphonate (Step F of FIG. 1). Analogously, an enzyme with phosphotrans-p-hydroxymethylbenzoylase activity interconverts p-hydroxymethyl benzoyl-CoA and (p-hydroxymethyl benzoyloxy)phosphonate (Step 0, FIG. 1). Enzymes with these activities have not been characterized to date. Exemplary phosphate-transferring acyltransferases include phosphotransacetylase (EC 2.3.1.8) and phosphotransbutyrylase (EC 2.3.1.19). The pta gene from E. coli encodes a phosphotransacetylase that reversibly converts acetyl-CoA into acetyl-phosphate (Suzuki, Biochim. Biophys. Acta 191:559-569 (1969)). This enzyme can also convert propionyl-CoA propionylphosphate (Hesslinger et al., Mol. Microbiol 27:477-492 (1998)). Other phosphate acetyltransferases that exhibit activity on propionyl-CoA are found in Bacillus subtilis (Rado et al., Biochim. Biophys. Acta 321:114-125 (1973)), Clostridium kluyveri (Stadtman, 1:596-599 (1955)), and Thermotoga maritima(Bock et al., J Bacteriol. 181:1861-1867 (1999)). Similarly, the ptb gene from C. acetobutylicum encodes phosphotransbutyrylase, an enzyme that reversibly converts butyryl-CoA into butyryl-phosphate (Wiesenborn et al., Appl Environ. Microbiol 55:317-322 (1989); Walter et al., Gene 134:107-111 (1993)). Additional ptb genes are found in butyrate-producing bacterium L2-50 (Louis et al., J Bacteriol. 186:2099-2106 (2004)) and Bacillus megaterium (Vazquez et al., Curr. Microbiol 42:345-349 (2001)).

| | GenBank | | |
|---|---|---|---|
| Gene | Accession No. | GI No. | Organism |
| pta | NP_416800.1 | 71152910 | Escherichia coli |
| pta | P39646 | 730415 | Bacillus subtilis |
| pta | A5N801 | 146346896 | Clostridium kluyveri |
| pta | Q9X0L4 | 6685776 | Thermotoga maritima |
| ptb | NP_349676 | 34540484 | Clostridium acetobutylicum |
| ptb | AAR19757.1 | 38425288 | butyrate-producing bacterium |
| | | | L2-50 |
| ptb | CAC07932.1 | 10046659 | Bacillus megaterium |

Kinase or phosphotransferase enzymes transform carboxylic acids to phosphonic acids with concurrent hydrolysis of one ATP. Such an enzyme is required to convert p-toluate to (p-methylbenzoyloxy)phosphonate (FIG. 1, Step E) and to transform p-hydroxymethyl benzoate to p-hydroxymethyl benzoyloxyphosphonate (FIG. 1, Step N). These exact transformations have not been demonstrated to date. Exemplary enzyme candidates include butyrate kinase (EC 2.7.2.7), isobutyrate kinase (EC 2.7.2.14), aspartokinase (EC 2.7.2.4), acetate kinase (EC 2.7.2.1) and gamma-glutamyl kinase (EC 2.7.2.11). Butyrate kinase catalyzes the reversible conversion of butyryl-phosphate to butyrate during acidogenesis in Clostridial species (Cary et al., Appl. Environ. Microbiol 56:1576-1583 (1990)). The Clostridium acetobutylicum enzyme is encoded by either of the two buk gene products (Huang et al., J Mol. Microbiol Biotechnol 2:33-38 (2000)). Other butyrate kinase enzymes are found in C. butyricum and C. tetanomorphum (TWAROG et al., J Bacterial. 86:112-117 (1963)). A related enzyme, isobutyrate kinase from Thermotoga maritima, was expressed in E. coli and crystallized (Diao et al., J Bacteriol. 191:2521-2529 (2009); Diao et al., Acta Crystallogr. D. Biol. Crystallogr. 59:1100-1102 (2003)). Aspartokinase catalyzes the ATP-dependent phosphorylation of aspartate and participates in the synthesis of several amino acids. The aspartokinase III enzyme in E. coli, encoded by lysC, has a broad substrate range and the catalytic residues involved in substrate specificity have been elucidated (Keng et al., Arch. Biochem. Biophys. 335:73-81 (1996)). Two additional kinases in E. coli are also good candidates: acetate kinase and gamma-glutamyl kinase. The E. coli acetate kinase, encoded by ackA (Skarstedt et al., J. Biol. Chem. 251:6775-6783 (1976)), phosphorylates propionate in addition to acetate (Hesslinger et al., Mol. Microbiol 27:477-492 (1998)). The E. coli gamma-glutamyl kinase, encoded by proB (Smith et al., J. Bacteriol. 157:545-551 (1984a)), phosphorylates the gamma carbonic acid group of glutamate.

| Protein | GenBank ID | GI Number | Organism |
|---|---|---|---|
| buk1 | NP_349675 | 15896326 | Clostridium acetobutylicum |
| buk2 | Q97II1 | 20137415 | Clostridium acetobutylicum |
| buk2 | Q9X278.1 | 6685256 | Thermotoga maritima |
| lysC | NP_418448.1 | 16131850 | Escherichia coli |
| ackA | NP_416799.1 | 16130231 | Escherichia coli |
| proB | NP_414777.1 | 16128228 | Escherichia coli |

CoA transferases catalyze the reversible transfer of a CoA moiety from one molecule to another. Steps D and L of FIG. 1 are catalyzed by enzymes with p-methylbenzoyl-CoA transferase and p-hydroxymethyl benzoyl-CoA transferase activities respectively. In these transformations, p-methylbenzoyl-CoA is formed from p-toluate and p-hydroxymethyl benzoyl-CoA is formed from p-hydroxymethyl benzoate by the transfer of the CoA group from a CoA donor such as acetyl-CoA, succinyl-CoA or others. Exemplary CoA transferase enzymes that react with similar substrates include cinnamoyl-CoA transferase (EC 2.8.3.17) and benzylsuccinyl-CoA transferase. Cinnamoyl-CoA transferase, encoded fldA in Clostridium sporogenes, transfers a CoA moiety from cinnamoyl-CoA to a variety of aromatic acid substrates including phenylacetate, 3-phenylpropionate and 4-phenylbutyrate (Dickert et al., Eur. J Biochem. 267:3874-3884 (2000)). Benzylsuccinyl-CoA transferase utilizes succinyl-CoA or maleyl-CoA as the CoA donor, forming benzylsuccinyl-CoA from benzylsuccinate. This enzyme was characterized in the denitrifying bacteria Thauera aromatica, where it is encoded by bbsEF (Leutwein et al., J Bacteriol. 183:4288-4295 (2001)).

| Gene | GenBank Accession No. | GI No. | Organism |
|---|---|---|---|
| fldA | AAL18808.1 | 16417587 | Clostridium sporogenes |
| bbsE | AAF89840.1 | 9622535 | Thauera aromatica |
| bbsF | AAF89841.1 | 9622536 | Thauera aromatica |

Additional candidate CoA transferase enzymes with diverse substrate ranges include succinyl-CoA transferase, 4-hydroxybutyryl-CoA transferase, butyryl-CoA transferase, glutaconyl-CoA transferase and acetoacetyl-CoA transferase. The gene products of call, cat2, and cat3 of Clostridium kluyveri have been shown to exhibit succinyl-CoA, 4-hydroxybutyryl-CoA, and butyryl-CoA transferase activity, respectively (Seedorf et al., Proc. Natl. Acad. Sci U.S.A 105:2128-2133 (2008); Sohling et al., J Bacteriol. 178:871-880 (1996b)). Similar CoA transferase activities are also present in Trichomonas vaginalis (van Grinsven et al., J. Biol. Chem. 283:1411-1418 (2008)) and Trypanosoma brucei (Riviere et al., J. Biol. Chem. 279:45337-45346 (2004)). The glutaconyl-CoA-transferase (EC 2.8.3.12) enzyme from anaerobic bacterium Acidaminococcus fermentans reacts with glutaconyl-CoA and 3-butenoyl-CoA (Mack et al., Eur. J. Biochem. 226:41-51 (1994)). The genes encoding this enzyme are gctA and gctB. This enzyme has reduced but detectable activity with other CoA derivatives including glutaryl-CoA, 2-hydroxyglutaryl-CoA, adipyl-CoA, crotonyl-CoA and acrylyl-CoA (Buckel et al., Eur. J Biochem. 118:315-321 (1981)). The enzyme has been cloned and expressed in E. coli (Mack et al., Eur. J. Biochem. 226:41-51 (1994)). Glutaconate CoA-transferase activity has also been detected in Clostridium sporosphaeroides and Clostridium symbiosum. Acetoacetyl-CoA transferase utilizes acetyl-CoA as the CoA donor is. This enzyme is encoded by the E. coli atoA (alpha subunit) and atoD (beta subunit) genes (Korolev et al., Acta Crystallogr. D. Biol. Crystallogr. 58:2116-2121 (2002); Vanderwinkel et al., Biochem. Biophys. Res. Commun. 33:902-908 (1968)). This enzyme has a broad substrate range (Sramek et al. Arch. Biochem. Biophys. 171-14-26, (1975)) and has been shown to transfer the CoA moiety from acetyl-CoA to a variety of substrates, including isobutyrate (Matthies et al., Appl Environ. Microbiol 58:1435-1439 (1992)), valerate (Vanderwinkel et al., Biochem. Biophys. Res. Commun. 33:902-908 (1968)) and butanoate (Vanderwinkel et al., Biochem. Biophys. Res. Commun. 33:902-908 (1968)). Similar enzymes exist in Corynebacterium glutamicum ATCC 13032 (Duncan et al., Appl. Environ. Microbiol 68:5186-5190 (2002)), Clostridium acetobutylicum (Cary et al., Appl. Environ. Microbiol 56:1576-1583 (1990); Wiesenborn et al., Appl. Environ. Microbiol 55:323-329 (1989)), and Clostridium saccharoperbutylacetonicum (Kosaka et al., Biosci. Biotechnol Biochem. 71:58-68 (2007)).

| | GenBank Accession | | |
|---|---|---|---|
| Gene | No | GI No. | Organism |
| cat1 | P38946.1 | 729048 | Clostridium kluyveri |
| cat2 | P38942.2 | 172046066 | Clostridium kluyveri |
| cat3 | EDK35586.1 | 146349050 | Clostridium kluyveri |
| TVAG_395550 | XP_001330176 | 123975034 | Trichomonas vaginalis G3 |
| Tb11.02.0290 | XP_828352 | 71754875 | Trypanosoma brucei |
| gctA | CAA57199.1 | 559392 | Acidaminococcus fermentans |
| gctB | CAA57200.1 | 559393 | Acidaminococcus fermentans |
| gctA | ACJ24333.1 | 212292816 | Clostridium symbiosum |
| gctB | ACJ24326.1 | 212292808 | Clostridium symbiosum |
| atoA | P76459.1 | 2492994 | Escherichia coli K12 |
| atoD | P76458.1 | 2492990 | Escherichia coli K12 |
| actA | YP_226809.1 | 62391407 | Corynebacterium glutamicum |
| cg0592 | YP_224801.1 | 62389399 | Corynebacterium glutamicum |
| ctfA | NP_149326.1 | 15004866 | Clostridium acetobutylicum |
| ctfB | NP_149327.1 | 15004867 | Clostridium acetobutylicum |
| ctfA | AAP42564.1 | 31075384 | Clostridium saccharoperbutylacetonicum |
| ctfB | AAP42565.1 | 31075385 | Clostridium saccharoperbutylacetonicum |

p-methylbenzoyl-CoA and p-hydroxymethyl-benzoyl-CoA can be hydrolyzed to their corresponding acids by CoA hydrolases or thioesterases in the EC class 3.1.2 (Steps D and L of FIG. 1). Exemplary CoA thioesters that hydrolyze benzoyl-CoA and/or similar substrates include 4-hydroxybenzoyl-CoA hydrolase (EC 3.1.2.23) and phenylglyoxal-CoA hydrolase (EC 3.1.2.25). The Azoarcus evansii gene orfl encodes an enzyme with benzoyl-CoA hydrolase activity that participates in benzoate metabolism (Ismail, Arch. Microbiol. 190:451-460 (2008)). This enzyme, when heterologously expressed in E. coli, demonstrated activity on a number of alternate substrates. Additional benzoyl-CoA enzyme candidates were identified in benzonate degradation gene clusters of Magnetospirillum magnetotacticum, Jannaschia sp. CCS1 and Sagittula stellata E-37 by sequence similarity (Ismail, Arch. Microbiol 190:451-460 (2008)). The 4-hydroxybenzoyl-CoA hydrolase of Pseudomonas sp. CBS3 accepts benzoyl-CoA and p-methylbenzoyl-CoA as substrates and has been heterologously expressed and characterized in E. coli (Song et al., Bioorg. Chem. 35:1-10 (2007)). Additional enzymes with demonstrated benzoyl-CoA hydrolase activity include the palmitoyl-CoA hydrolase of Mycobacterium tuberculosis (Wang et al., Chem. Biol. 14:543-551 (2007)) and the acyl-CoA hydrolase of E. coli encoded by entH (Guo et al., Biochemistry 48:1712-1722 (2009)).

| | GenBank Accession | | |
|---|---|---|---|
| Gene | No. | GI No. | Organism |
| orf1 | AAN39365.1 | 23664428 | Azoarcus evansii |
| Magn03011230 | ZP_00207794 | 46200680 | Magnetospirillum |
| | | | magnetotacticum |
| Jann_0674 | YP_508616 | 89053165 | Jannaschia sp. CCS1 |
| SSE37_24444 | ZP_01745221 | 126729407 | Sagittula stellata |
| EF569604.1:4745 . . . 5170 | ABQ44580.1 | 146761194 | Pseudomonas sp. CBS3 |
| Rv0098 | NP_214612.1 | 15607240 | Mycobacterium tuberculosis |
| entH | AAC73698.1 | 1786813 | Escherichia coli |

Several CoA hydrolases with broad substrate ranges represent suitable candidate enzymes for hydrolyzing p-methylbenzoyl-CoA and/or p-hydroxymethyl benzoyl-CoA. For example, the enzyme encoded by acot12 from Rattus norvegicus brain (Robinson et al., Biochem. Biophys. Res. Commun. 71:959-965 (1976)) can react with butyryl-CoA, hexanoyl-CoA and malonyl-CoA. The human dicarboxylic acid thioesterase, encoded by acot8, exhibits activity on glutaryl-CoA, adipyl-CoA, suberyl-CoA, sebacyl-CoA, and dodecanedioyl-CoA (Westin et al., J. Biol. Chem. 280:38125-38132 (2005)). The closest E. coli homolog to this enzyme, tesB, can also hydrolyze a range of CoA thiolesters (Naggert et al., J Biol Chem 266:11044-11050 (1991)). A similar enzyme has also been characterized in the rat liver (Deana R., Biochem Int 26:767-773 (1992)).

| Gene name | GI# | GenBank Accession # | Organism |
|---|---|---|---|
| acot12 | 18543355 | NP_570103.1 | Rattus norvegicus |
| tesB | 16128437 | NP_414986 | Escherichia coli |
| acot8 | 3191970 | CAA15502 | Homo sapiens |
| acot8 | 51036669 | NP_570112 | Rattus norvegicus |
| tesA | 16128478 | NP_415027 | Escherichia coli |
| ybgC | 16128711 | NP_415264 | Escherichia coli |
| paaI | 16129357 | NP_415914 | Escherichia coli |
| ybdB | 16128580 | NP_415129 | Escherichia coli |

The ATP-dependent activation of p-toluate to p-methylbenzoyl-CoA (Step D of FIG. 1) and of p-hydroxymethyl benzoate to p-hydroxymethyl benzoyl-CoA (Step L of FIG. 1) is catalyzed by a CoA synthetase or acid-thiol ligase. AMP-forming CoA ligases activate the aromatic acids to their corresponding CoA derivatives, whereas ADP-forming CoA ligases are generally reversible. Exemplary AMP-forming benzoyl-CoA ligases from Thauera aromatics and Azoarcus sp. strain CIB have been characterized (Lopez Barragan et al., J Bacteriol. 186:5762-5774 (2004); Schuhle et al., J Bacteriol. 185:4920-4929 (2003)). Alternately, AMP-forming CoA ligases that react with structurally similar substrates may have activity on benzoate or p-toluate. The AMP-forming cyclohexanecarboxylate CoA-ligase from Rhodopseudomonas palustris, encoded by aliA, is well-characterized, and alteration of the active site has been shown to impact the substrate specificity of the enzyme (Samanta et al., Mol. Microbiol 55:1151-1159 (2005)). This enzyme also functions as a cyclohex-1-ene-1-carboxylate CoA-ligase during anaerobic benzene ring degradation (Egland et al., Proc. Natl. Acad. Sci U.S.A 94:6484-6489 (1997)). Additional exemplary CoA ligases include two characterized phenylacetate-CoA ligases from P. chrysogenum (Lamas-Maceiras et al., Biochem. J 395:147-155 (2006); Wang et al., Biochem. Biophys. Res. Commun. 360:453-458 (2007)); Wang et al., Biochem. Biophys. Res. Commun. 360:453-458 (2007)), the phenylacetate-CoA ligase from Pseudomonas putida (Martinez-Blanco et al., J Biol. Chem. 265:7084-7090 (1990)), and the 6-carboxyhexanoate-CoA ligase from Bacillus subtilis (Bower et al., J Bacteriol. 178:4122-4130 (1996)).

| | GenBank | | |
|---|---|---|---|
| Gene | Accession No. | GI No. | Organism |
| bclA | Q8GQN9.1 | 75526585 | Thauera aromatica |
| bzdA | AAQ08820.1 | 45649073 | Azoarcus sp. strain CIB |
| aliA | AAC23919 | 2190573 | Rhodopseudomonas palustris |
| phl | CAJ15517.1 | 77019264 | Penicillium chrysogenum |
| phlB | ABS19624.1 | 152002983 | Penicillium chrysogenum |
| paaF | AAC24333.2 | 22711873 | Pseudomonas putida |
| bioW | NP_390902.2 | 50812281 | Bacillus subtilis |

ADP-forming CoA ligases catalyzing this exact transformation have not been characterized to date; however, several enzymes with broad substrate specificities have been described in the literature. The ADP-forming acetyl-CoA synthetase (ACD, EC 6.2.1.13) from Archaeoglobus fulgidus, encoded by AF1211, was shown to operate on a variety of linear and branched-chain substrates including isobutyrate, isopentanoate, and fumarate (Musfeldt et al., J Bacteriol. 184:636-644 (2002)). A second reversible ACD in Archaeoglobus fulgidus, encoded by AF1983, was also shown to have abroad substrate range with high activity on aromatic compounds phenylacetate and indoleacetate (Musfeldt et al., supra). The enzyme from Haloarcula marismortui, annotated as a succinyl-CoA synthetase, accepts propionate, butyrate, and branched-chain acids (isovalerate and isobutyrate) as substrates, and was shown to operate in the forward and reverse directions (Brasen et al., Arch. Microbiol 182:277-287 (2004)). The ACD encoded by PAE3250 from hyperthermophilic crenarchaeon Pyrobaculum aerophilum showed the broadest substrate range of all characterized ACDs, reacting with acetyl-CoA, isobutyryl-CoA (preferred substrate) and phenylacetyl-CoA (Brasen and Schonheit, Arch. Microbiol 182:277-287 (2004)). Directed evolution or engineering can be used to modify this enzyme to operate at the physiological temperature of the host organism. The enzymes from A. fulgidus, H. marismortui and P. aerophilum have all been cloned, functionally expressed, and characterized in E. coli (Brasen and Schonheit, Arch. Microbiol 182:277-287 (2004); Musfeldt and Schonheit, J Bacteriol. 184:636-644 (2002)). An additional candidate is the enzyme encoded by sucCD in E. coli, which naturally catalyzes the formation of succinyl-CoA from succinate with the concomitant consumption of one ATP, a reaction which is reversible in vivo (Buck et al., Biochemistry 24:6245-6252 (1985)). The acyl CoA ligase from Pseudomonas putida has been demonstrated to work on several aliphatic substrates including acetic, propionic, butyric, valerie, hexanoic, heptanoic, and octanoic acids and on aromatic compounds such as phenylacetic and phenoxyacetic acids (Fernandez-Valverde et al., Appl. Environ. Microbiol. 59:1149-1154 (1993)). A related enzyme, malonyl CoA synthetase (6.3.4.9) from Rhizobium leguminosarum could convert several diacids, namely, ethyl-, propyl-, allyl-, isopropyl-, dimethyl-, cyclopropyl-, cyclopropylmethylene-, cyclobutyl-, and benzyl-malonate into their corresponding monothioesters (Pohl et al., J. Am. Chem. Soc. 123:5822-5823 (2001)).

| | GenBank | | |
|---|---|---|---|
| | Accession | | |
| Gene | No. | GI No. | Organism |
| AF1211 | NP_070039.1 | 11498810 | Archaeoglobus fulgidus |
| AF1983 | NP_070807.1 | 11499565 | Archaeoglobus fulgidus |
| Scs | YP_135572.1 | 55377722 | Haloarcula marismortui |
| PAE3250 | NP_560604.1 | 18313937 | Pyrobaculum aerophilum str. |
| | | | IM2 |
| sucC | NP_415256.1 | 16128703 | Escherichia coli |
| sucD | AAC73823.1 | 1786949 | Escherichia coli |
| paaF | AAC24333.2 | 22711873 | Pseudomonas putida |
| matB | AAC83455.1 | 3982573 | Rhizobium leguminosarum |

Step A (1.3.99.15) in FIG. 2 shows the conversion of 4-hydroxymethyl benzoyl-CoA into 4-hydroxymethyl-cyclohex-1,5-diene carboxyl-CoA via p-hydroxymethyl benzoyl-CoA reductase. Although enzymes that can catalyze this exact transformation are not known, an exemplary candidate is benzoyl-CoA reductase from Rhodopseudomonas palustris. This enzyme converts benzoyl-CoA into cyclohex-1,5-diene carboxyl-CoA. Benzoyl-CoA reductase catalyzes the two-electron ring reduction of benzoyl-CoA to a cyclic dienoyl-CoA. This reaction is driven by the hydrolysis of 2 molecules of ATP to ADP+Pi. The genes badDEFG encode for the enzyme benzoyl-CoA reductase in Rhodopseudomonas palustris (Egland, P. G., et al., Proc Natl Acad Sci USA 94:6484-6489 (1997)). The genes badDEFG are transcribed in an operon and their expression is induced by anaerobic growth on benzoate. BcrCBAD from Thauera aromatica also possesses a benzoyl-CoA reductase that transforms benzoyl-CoA into cyclohex-1,5-diene-1-carboxyl-CoA (Breese, K., et al., Eur J Biochem. 256:148-154 (1998)). The genes from Azoarcus involved in the reduction of benzoyl-CoA to cyclohex-1,5-diene carboxyl-CoA are also arranged in a cluster (Carmona, M., et al., Mol Microbiol 58:1210-1215 (2005)).

| | GenBank Accession | | |
|---|---|---|---|
| Gene | No. | GI No. | Organism |
| badD | NP_946010.1 | 39933734 | Rhodopseudomonas palustris |
| badE | NP_946011.1 | 39933735 | Rhodopseudomonas palustris |
| badF | NP_946012.1 | 39933736 | Rhodopseudomonas palustris |
| badG | NP_946013.1 | 39933737 | Rhodopseudomonas palustris |
| bcrC | CAA12247.1 | 3724168 | Thauera aromatica |
| bcrB | CAA12248.1 | 3724169 | Thauera aromatica |
| bcrA | CAA12249.1 | 3724170 | Thauera aromatica |
| bcrD | CAA12250.1 | 3724171 | Thauera aromatica |
| bzdN | AAQ08806.1 | 45649067 | Azoarcus sp. CIB |
| bzdO | AAQ08807.1 | 45649068 | Azoarcus sp. CIB |
| bzdP | AAQ08808.1 | 33326777 | Azoarcus sp. CIB |
| bzdQ | AAQ08809.1 | 45649069 | Azoarcus sp. CIB |

The reduction of 4-hydroxymethyl-cyclohex-1,5-diene carboxyl-CoA into 4-hydroxymethyl cyclohex-1-ene carboxyl-CoA (Step B, FIG. 2) has not been described in literature. The exact enzyme catalyzing this conversion has not been identified. However, it has been reported that benzoyl-CoA reductase from R. palustris can catalyze the reduction of benzoyl-CoA to cyclohex-1,5-diene carboxyl-CoA and further to cyclohex-1-ene carboxyl-CoA. This enzyme is therefore a good candidate for the reduction of 4-hydroxymethyl-cyclohex-1,5-diene carboxyl-CoA into 4-hydroxymethyl cyclohex-1-ene carboxyl-CoA (Carmona, M., et al., Mol Microbiol 58:1210-1215 (2005)).

| | Gene Bank Accession | | |
|---|---|---|---|
| Gene | No. | GI No. | Organism |
| badD | NP_946010.1 | 39933734 | Rhodopseudomonas palustris |
| badE | NP_946011.1 | 39933735 | Rhodopseudomonas palustris |
| badF | NP_946012.1 | 39933736 | Rhodopseudomonas palustris |
| badG | NP_946013.1 | 39933737 | Rhodopseudomonas palustris |

4-hydroxymethyl cyclohex-1-ene carboxyl-CoA can be reduced to 4-hydroxymethyl cyclohexanoyl-CoA (Step C. FIG. 2) by a dehydrogenase. One exemplary candidate is cyclohexanecarboxyl-CoA dehydrogenase from R. palustris encoded by aliB. Other candidates are found by sequence homology to aliB.

| | GenBank Accession | | |
|---|---|---|---|
| Gene | No. | GI No. | Organism |
| aliB | NP_946005.1 | 39933729 | Rhodopseudomonas |
| | | | palustris |
| 1UKW_A | 1UKW_A | 56966249 | Thermus thermophilus |
| 2PG0_A | 2PG0_A | 149243125 | Geobacillus kaustophilus |

Enoyl-CoA reductase enzymes are also suitable enzymes for catalyzing the reduction of 4-hydroxymethyl cyclohex-1-ene carboxyl-CoA to 4-hydroxymethyl cyclohexanoly-CoA (FIG. 2, Step C). One exemplary enoyl-CoA reductase is the gene product of bcd from C. acetobutylicum (Atsumi, S., et al., Metab Eng 10:305-311 (2008)), (Boynton, Z. L., et al., J Bacteriol. 178:3015-3024 (1996)), which naturally catalyzes the reduction of crotonyl-CoA to butyryl-CoA. Activity of this enzyme can be enhanced by expressing bcd in conjunction with expression of the C. acetobutylicum etfAB genes, which encode an electron transfer flavoprotein. An additional candidate for the enoyl-CoA reductase step is the mitochondrial enoyl-CoA reductase from E. gracilis (Hoffmeister, M., et al., J Biol. Chem. 280:4329-4338 (2005)). A construct derived from this sequence following the removal of its mitochondrial targeting leader sequence was cloned in E. coli resulting in an active enzyme (Hoffmeister, M., et al., J Biol. Chem. 280:4329-4338 (2005)). This approach is well known to those skilled in the art of expressing eukaryotic genes, particularly those with leader sequences that may target the gene product to a specific intracellular compartment, in prokaryotic organisms. A close homolog of this gene, TDE0597 from the prokaryote Treponema denticola, represents a third enoyl-CoA reductase which has been cloned and expressed in E. coli (Tucci, S., et al., Febs Letters 581:1561-1566 (2007)).

| | | GenBank | |
|---|---|---|---|
| Gene name | GI# | Accession # | Organism |
| Bcd | 15895968 | NP_349317.1 | Clostridium acetobutylicum |
| etfA | 15895966 | NP_349315.1 | Clostridium acetobutylicum |
| etfB | 15895967 | NP_349316.1 | Clostridium acetobutylicum |
| TER | 62287512 | Q5EU90.1 | Euglena gracilis |
| TDE0597 | 42526113 | NP_971211.1 | Treponema denticola |

Additional enoyl-CoA reductase enzyme candidates are found in organisms that degrade aromatic compounds. Rhodopseudomonas palustris, a model organism for benzoate degradation, has the enzymatic capability to degrade pimelate via beta-oxidation of pimeloyl-CoA. Adjacent genes in the pim operon, pimC and pimD, bear sequence homology to C. acetobutylicum bcd and are predicted to encode a flavin-containing pimeloyl-CoA dehydrogenase (Harrison, F. H., et al., Microbiology 151:727-736 (2005)). The genome of nitrogen-fixing soybean symbiont Bradyrhizobium japonicum also contains a pim operon composed of genes with high sequence similarity to pimC and pimD of R. palustris (Harrison, F. H., et al., Microbiology 151:727-736 (2005)).

| | | GenBank | |
|---|---|---|---|
| Gene name | GI# | Accession # | Organism |
| pimC | 39650632 | CAE29155 | Rhodopseudomonas palustris |
| pimD | 39650631 | CAE29154 | Rhodopseudomonas palustris |
| pimC | 27356102 | BAC53083 | Bradyrhizobium japonicum |
| pimD | 27356101 | BAC53082 | Bradyrhizobium japonicum |

An additional candidate is 2-methyl-branched chain enoyl-CoA reductase (EC 1.3.1.52), an enzyme catalyzing the reduction of sterically hindered trans-enoyl-CoA substrates. This enzyme participates in branched-chain fatty acid synthesis in the nematode Ascarius suum and is capable of reducing a variety of linear and branched chain substrates including 2-methylbutanoyl-CoA, 2-methylpentanoyl-CoA, octanoyl-CoA and pentanoyl-CoA (Duran, E., et al., J Biol. Chem. 268:22391-22396 (1993)). Two isoforms of the enzyme, encoded by genes acadl and acad, have been characterized.

| Gene name | GI# | GenBank Accession # | Organism |
|---|---|---|---|
| acad1 | 2407655 | AAC48316.1 | Ascarius suum |
| acad | 347404 | AAA16096.1 | Ascarius suum |

Step D in FIG. 2 shows the transformation of 4-hydroxymethyl cyclohexanoyl-CoA into 4-hydroxymethyl cyclohexane carbaldehyde via 4-hydroxymethyl cyclohexanoyl-CoA reductase. While enzymes catalyzing these exact transformations have not been reported yet, such a reaction can be catalyzed by acyl-CoA reductases that reduce an acyl-CoA to its corresponding aldehyde (Enzyme class 1.2.1b). Exemplary enzymes include fatty acyl-CoA reductase (EC 1.2.1.50), succinyl-CoA reductase (EC 1.2.1.76), acetyl-CoA reductase (EC 1.2.1.10) and butyryl-CoA reductase. Exemplary fatty acyl-CoA reductases enzymes are encoded by acr1 of Acinetobacter calcoaceticus (Reiser et al., 179:2969-2975 (1997)) and Acinetobacter sp. M-1 (Ishige et al., Appl. Environ. Microbiol. 68:1192-1195 (2002)). Enzymes with succinyl-CoA reductase activity are encoded by sucD of Clostridium kluyveri (Sohling et al., J Bacteriol. 178:871-880 (1996a); Sohling et al., 178:871-80 (1996)) and sucD of P. gingivalis (Takahashi et al., J Bacteriol. 182:4704-4710 (2000)). Additional succinyl-CoA reductase enzymes participate in the 3-hydroxypropionate/4-hydroxybutyrate cycle of thermophilic archaea such as Metallosphaera sedula (Berg et al., Science. 318:1782-1786 (2007)) and Thermoproteus neutrophilus (Ramos-Vera et al., J Bacteriol. 191:4286-4297 (2009)). The M. sedula CoA reductase, encoded by Msed 0709, is strictly NADPH-dependent and also has malonyl-CoA reductase activity. The T. neutrophilus enzyme is active with both NADPH and NADH. The acylating acetaldehyde dehydrogenase in Pseudomonas sp, encoded by bphG, has been demonstrated to oxidize and acylate acetaldehyde, propionaldehyde, butyraldehyde, isobutyraldehyde and formaldehyde to their corresponding CoA-esters (Powlowski et al., 175:377-385 (1993)). In addition to reducing acetyl-CoA to ethanol, the enzyme encoded by adhE in Leuconostoc mesenteroides has been shown to oxidize the branched chain compound isobutyraldehyde to isobutyryl-CoA (Koo et al., Biotechnol Lett. 27:505-510 (2005)). Butyraldehyde dehydrogenase catalyzes a similar reaction, conversion of butyryl-CoA to butyraldehyde, in solventogenic organisms such as Clostridium saccharoperbutylacetonicum (Kosaka et al., Biosci. Biotechnol Biochem. 71:58-68 (2007)). The acyl-CoA reductase encoded by ald in Clostridium beijerinckii has been reported to reduce acetyl-CoA and butyryl-CoA to their corresponding aldehydes (Toth et al., Appl Environ. Microbiol 65:4973-4980 (1999)). This enzyme exhibits high sequence homology to the CoA-dependent acetaldehyde dehydrogenase enzymes of Salmonella typhimurium and E. coli encoded by eutE (Toth et al., Appl Environ. Microbiol 65:4973-4980 (1999)).

| | GenBank | | |
|---|---|---|---|
| Gene | Accession No. | GI No. | Organism |
| acr1 | YP_047869.1 | 50086359 | Acinetobacter |
| | | | calcoaceticus |
| acr1 | AAC45217 | 1684886 | Acinetobacter baylyi |
| acr1 | BAB85476.1 | 18857901 | Acinetobacter sp. |
| | | | Strain M-1 |
| MSED_0709 | YP_001190808.1 | 146303492 | Metallosphaera sedula |
| Tneu_0421 | ACB39369.1 | 170934108 | Thermoproteus |
| | | | neutrophilus |
| sucD | P38947.1 | 172046062 | Clostridium kluyveri |
| sucD | NP_904963.1 | 34540484 | Porphyromonas |
| | | | gingivalis |
| bphG | BAA03892.1 | 425213 | Pseudomonas sp |
| adhE | AAV66076.1 | 55818563 | Leuconostoc |
| | | | mesenteroides |
| bld | AAP42563.1 | 31075383 | Clostridium |
| | | | saccharoperbutyl- |
| | | | acetonicum |
| ald | AAT66436 | 9473535 | Clostridium |
| | | | beijerinckii |
| eutE | AAA80209 | 687645 | Salmonella |
| | | | typhimurium |
| eutE | P77445 | 2498347 | Escherichia coli |

An additional CoA reductase enzyme candidate is malonyl-CoA reductase which transforms malonyl-CoA to malonic semialdehyde. Malonyl-CoA reductase is a key enzyme in autotrophic carbon fixation via the 3-hydroxypropionate cycle in thermoacidophilic archael bacteria (Berg et al., 318:1782-1786 (2007); Thauer, 318:1732-1733 (2007)). The enzyme utilizes NADPH as a cofactor and has been characterized in Metallosphaera and Sulfolobus spp (Alber et al., 188:8551-8559 (2006); Hugler et al., 184:2404-2410 (2002)). The enzyme is encoded by Msed 0709 in Metallosphaera sedula (Alber et al., 188:8551-8559 (2006); Berg et al., 318:1782-1786 (2007)). A gene encoding a malonyl-CoA reductase from Sulfolobus tokodaii was cloned and heterologously expressed in E. coli (Alber et al., 188:8551-8559 (2006)). This enzyme has also been shown to catalyze the conversion of methylmalonyl-CoA to its corresponding aldehyde (WO/2007/141208). Both malonyl-CoA reductase enzyme candidates have high sequence similarity to aspartate-semialdehyde dehydrogenase, an enzyme catalyzing the reduction and concurrent dephosphorylation of aspartyl-4-phosphate to aspartate semialdehyde. Additional gene candidates can be found by sequence homology to proteins in other organisms including Sulfolobus solfataricus and Sulfolobus acidocaldarius.

| | GenBank Accession | | |
|---|---|---|---|
| Gene | No. | GI No. | Organism |
| MSED_0709 | YP_001190808.1 | 146303492 | Metallosphaera |
| | | | sedula |
| mcr | NP_378167.1 | 15922498 | Sulfolobus tokodaii |
| asd-2 | NP_343563.1 | 15898958 | Sulfolobus |
| | | | solfataricus |
| Saci_2370 | YP_256941.1 | 70608071 | Sulfolobus |
| | | | acidocaldarius |

Step E, FIG. 2 shows the reduction of 4-hydroxymethyl cycloehexanoyl-CoA into 1,4-cyclohexanedimethanol (Enzyme class 1.1.1.a). Exemplary genes encoding enzymes that catalyze the conversion of an aldehyde to alcohol (i.e., alcohol dehydrogenase or equivalently aldehyde reductase) include alrA encoding a medium-chain alcohol dehydrogenase for C2-C14 (Tani, A., et al., Appl. Environ. Microbiol. 66:5231-5235 (2000)), yqhD from E. coli which has preference for molecules longer than C(3) (Sulzenbacher, G., Journal of Molecular Biology 342:489-502 (2004)), and bdh I and bdh II from C. acetobutylicum which converts butyryaldehyde into butanol (Walter, K. A., et al., Journal of Bacteriology 174:7149-7158 (1992)). The gene product of yqhD catalyzes the reduction of acetaldehyde, malondialdehyde, propionaldehyde, butyraldehyde, and acrolein using NADPH as the cofactor (Perez, J. M., et al., J Biol. Chem. 283:7346-7353 (2008)), (Perez, J. M., J Biol. Chem. 283:7346-7353 (2008)). The adhA gene product from Zymomonas mobilis has been demonstrated to have activity on a number of aldehydes including formaldehyde, acetaldehyde, propionaldehyde, butyraldehyde, and acrolein (Kinoshita, S., et al., Appl Microbiol Biotechnol 22:249-254 (1985)). Additional aldehyde reductase candidates are encoded by bdh in C. saccharoperbutylacetonicum and Cbei_{—}1722, Cbei_{—}2181 and Cbei_{—}2421 in C. beijerinckii.

| Gene | GenBank ID | GI number | Organism |
|---|---|---|---|
| alrA | BAB12273.1 | 9967138 | Acinetobacter sp. strain |
| | | | M-1 |
| yqhD | NP_417484.1 | 16130909 | Escherichia coli |
| bdh I | NP_349892.1 | 15896543 | Clostridium |
| | | | acetobutylicum |
| bdh II | NP_349891.1 | 15896542 | Clostridium |
| | | | acetobutylicum |
| adhA | YP_162971.1 | 56552132 | Zymomonas mobilis |
| bdh | BAF45463.1 | 124221917 | Clostridium |
| | | | saccharoperbutyl- |
| | | | acetonicum |
| Cbei_1722 | YP_001308850 | 150016596 | Clostridium beijerinckii |
| Cbei_2181 | YP_001309304 | 150017050 | Clostridium beijerinckii |
| Cbei_2421 | YP_001309535 | 150017281 | Clostridium beijerinckii |

Enzymes exhibiting 4-hydroxybutyrate dehydrogenase activity (EC 1.1.1.61) also fall into this category. Such enzymes have been characterized in Ralstonia eutropha (Bravo, D. T., et al., J. Forensic Sci. 49:379-387 (2004)), Clostridium kluyveri (Wolff, R. A., et al., Protein Expr. Purif. 6:206-212 (1995)) and Arabidopsis thaliana (Breitkreuz, K. E., et al., J. Biol. Chem. 278:41552-41556 (2003)). The A. thaliana enzyme was cloned and characterized in yeast (Breitkreuz, K. E., et al., J. Biol. Chem. 278:41552-41556 (2003)). Yet another gene is the alcohol dehydrogenase adhI from Geobacillus thermoglucosidasius (Jeon, Y. J., J Biotechnol 135:127-133 (2008)).

| Gene | GenBank ID | GI number | Organism |
|---|---|---|---|
| 4hbd | YP_726053.1 | 113867564 | Ralstonia eutropha H16 |
| 4hbd | L21902.1 | 146348486 | Clostridium kluyveri |
| | | | DSM 555 |
| 4hbd | Q94B07 | 75249805 | Arabidopsis thaliana |
| adhI | AAR91477.1 | 40795502 | Geobacillus |
| | | | thermoglucosidasius |

Another exemplary enzyme is 3-hydroxyisobutyrate dehydrogenase (EC 1.1.1.31) which catalyzes the reversible oxidation of 3-hydroxyisobutyrate to methylmalonate semialdehyde. This enzyme participates in valine, leucine and isoleucine degradation and has been identified in bacteria, eukaryotes, and mammals. The enzyme encoded by P84067 from Thermus thermophilus HB8 has been structurally characterized (Lokanath, N. K., et al., J Mol Biol 352:905-17. (2005)). The reversibility of the human 3-hydroxyisobutyrate dehydrogenase was demonstrated using isotopically-labeled substrate (Manning, N. J., et al., Biochem J 231:481-4, 1985)). Additional genes encoding this enzyme include 3hidh in Homo sapiens (Hawes, J. W., et al., Methods Enzymol. 324:218-228 (2000)) and Oryctolagus cuniculus (Chowdhury, E. K., et al., Biosci. Biotechnol Biochem. 60:2043-2047 (1996). (Hawes, J. W., Methods Enzymol. 324:218-228 (2000)), mmsB in Pseudomonas aeruginosa and Pseudomonas putida, and dhat in Pseudomonas putida (Aberhart, D. J., et al., J Chem. Soc. [Perkin 1] 6:1404-1406. (1979)). (Chowdhury, E. K., et al., Biosci. Biotechnol Biochem. 60:2043-2047 (1996)). (Chowdhury, E. K., et al., Biosci. Biotechnol Biochem. 60:2043-2047 (1996)). Several 3-hydroxyisobutyrate dehydrogenase enzymes have been characterized in the reductive direction, including mmsB from Pseudomonas aeruginosa (Gokarn, R. R., et al., U.S. Pat. No. 7,393,676, (2008)) and mmsB from Pseudomonas putida.

| Gene | GenBank ID | GI number | Organism |
|---|---|---|---|
| P84067 | P84067 | 75345323 | Thermus thermophilus |
| 3hidh | P31937.2 | 12643395 | Homo sapiens |
| 3hidh | P32185.1 | 416872 | Oryctolagus cuniculus |
| mmsB | NP_746775.1 | 26991350 | Pseudomonas putida |
| mmsB | P28811.1 | 127211 | Pseudomonas aeruginosa |
| dhat | Q59477.1 | 2842618 | Pseudomonas putida |

3-Hydroxypropionate dehydrogenase, also known as malonate semialdehyde reductase, catalyzes the reversible conversion of malonic semialdehyde to 3-HP. An NADH-dependent 3-hydroxypropionate dehydrogenase is thought to participate in beta-alanine biosynthesis pathways from propionate in bacteria and plants (Rathinasabapathi B., Journal of Plant Pathology 159:671-674 (2002), (Sulzenbacher, G., et al., Journal of Molecular Biology 342:489-502, (2004)). This enzyme has not been associated with a gene in any organism to date. An NADPH-dependent malonate semialdehyde reductase catalyzes the reverse reaction in autotrophic CO₂-fixing bacteria. Although the enzyme activity has been detected in Metallosphaera sedula, the identity of the gene is not known (Alber, B., et al., J. Bacteriol. 188:8551-8559 (2006)). Several 3-hydroxyisobutyrate dehydrogenase enzymes exhibit 3-hydroxypropionate dehydrogenase activity. Three genes exhibiting this activity are mmsB from Pseudomonas aeruginosa PAO1 (Gokarn, R. R., et al., U.S. Pat. No. 7,393,676, (2008)), mmsB from Pseudomonas putida KT2440 and mmsB from Pseudomonas putida E23 (Chowdhury, E. K., et al., Biosci. Biotechnol. Biochem. 60:2043-2047 (1996)).

| GENE | GENBANK ID | GI NUMBER | ORGANISM |
|---|---|---|---|
| mmsB | NP_252259.1 | 15598765 | Pseudomonas putida |
| mmsB | NP_746775.1 | 26991350 | Pseudomonas aeruginosa |
| mmsB | JC7926 | 60729613 | Pseudomonas putida |

Chemical reduction of dihydroxymethyl benzene can be accomplished by methods that have been reported to reduce the aromatic ring of benzene and other aromatic compounds. Selectivity for the reduction of the aromatic ring over the benzylic alcohol is known in the art. Conditions similar to those useful for the reduction of phenol to cyclohexanol is favored in the presence of Rhodium and ruthenium catalysts (Comprehensive organic functional group transformations. Katritzky et al, Pergamon, (1995)). The reduction of 1-naphthol to decanol proceeds in high yields of 94-97% using a rhodium catalyst (5% Rh/alumina in ethanol) at pressures of 381-410 KPa for 12 hours (The first equation below). Ruthenium (RuO₂) requires higher pressure but can accomplish the reduction of phenols to cyclohexanols at high yields (The second equation below). Similar conditions can be applied to selectively reduce benzylic alcohols by varying the permittivity of the solvent (Takagi et al. Energy & Fuels, 13, 1191-1196, (1999)). Raney Ni_Al alloy allows the efficient saturation of phenol in dilute aqueous alkaline solution without any solvent as shown in the third equation below (T. Tsukinoki, T. Kanda, G.-B. Liu, H. Tsuzuki, M. Tashiro, Tetrahedron Lett. 2000, 41)5865-5868.) A stereoselective hydrogenation of 4-aminobenzoic acid has been reported with Rh/Al2O3 as catalyst (Windhorst et al. J. Fluorine Chem. 1996, 80, 35-40.)

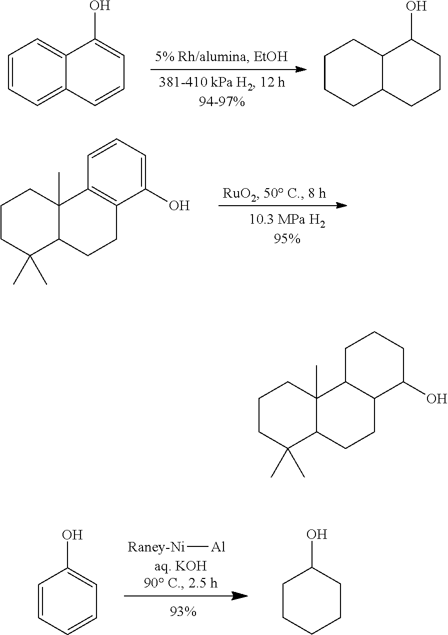

Other methods of catalytic reduction of the benzene ring include Benseker reduction that uses Li in ethylene diamine as the catalyst has been demonstrated to reduce benzene into cyclohexane. Cyclohexane can also be obtained from benzene with 0.01 molar equivalents of the cobalt complex at 20° C. and 1 atmospheric pressure of hydrogen (Comprehensive organic functional group transformations, Katritzky et al, Pergamon, (1995)). Hydrogenations using the catalyst were reported to be stereospecific for only cis-products. This enzyme was shown to work on naphthalene that was reduced to give cis-decalin as the sole product. Rhodium catalysts have also been employed to reduce benzene into cyclohexane under conditions of room temperature and atmospheric pressure of hydrogen. A Ruthenium-based catalyst (C₆Me₆)₂Ru has also been reported to reduce 1,4-dimethylbenzene into cyclohexane and cyclohexene derivatives under elevated temperature and pressure. Another way to reduce benzene ring is by the Birch reaction in which hydrogen is added to the benzene ring by treatment with the electron rich solution of alkali metals, usually lithium or sodium, in liquid ammonia. This reaction takes place at room temperature and atmospheric pressure. High yields of 1,4-cyclohexadiene (80-90%) are obtained (Altundas, et al., Turk J Chem 29:513-518 (2005) and this can be further reduced to cyclohexane with a mild oxidant. The same reduction process can be applied to benzene rings substituted with electron-donating groups such as alkyl, alkoxy and amino groups with a slight modification that alcohol should be present in the reaction mixture because ammonia is not a strong enough acid to protonate the intermediates Activated benzenes such as those with carbonyl groups, biphenyl and smaller polycyclic aromatics do not require alcohol for reduction.

Phase transfer systems involving aqueous RhCl₃ and Aliquat 336 and 1,2-dichloroethane in the organic phase have also been used to reduce aromatic compounds at temperature of 30° C. and atmospheric pressure with 51% conversion of benzene into cyclohexane with no other products (Comprehensive organic functional group transformations, Katritzky et al Pergamon, (1995)).

The reduction of benzene can be achieved by using either LiBr+HMPA (Hexamethylphosphoramide) as a solvent (water as proton donor) or in ethanol+HMPA at an aluminum cathode. Reduction of other aromatic compounds has also been achieved by a similar process (Marcel Dukker, Organic Electrochemistry. (2001)).

Liquid-phase hydrogenation processes are operated in several commercial plants for example by Mitsubishi Chemical, Hydrocarbon-Sinclair, and IFP. In the IFP process, a Raney nickel catalyst is employed in a bubble column reactor at 200°-225° C. and 50 bar. The nickel suspension is circulated to improve heat removal, the benzene being completely hydrogenated in a coupled fixed-bed reactor. Numerous gas-phase hydrogenations have also been developed e.g. by Arco, DSM, Toray, Houndry, and UOP. The Hydrar-process (UOP) uses a series of three reactors with a Ni/support and a stepwise increasing temperature (400°-600° C.) at 30 bars. In the Arco process a noble metal catalyst is used by which conversion of benzene is achieved. Kinetic studies of gas-phase benzene hydrogenation over supported nickel, palladium or platinum catalysts have been reported (U.S. Pat. No. 4,731,496. March 1988).

Throughout this application various publications have been referenced. The disclosures of these publications in their entireties, including GenBank and GI number publications, are hereby incorporated by reference in this application in order to more fully describe the state of the art to which this invention pertains. Although the invention has been described with reference to the examples provided above, it should be understood that various modifications can be made without departing from the spirit of the invention.

1. A non-naturally occurring microbial organism having a 1,4-cyclohexanedimethanol pathway, wherein said microbial organism comprises at least one exogenous nucleic acid encoding a 1,4-cyclohexanedimethanol pathway enzyme expressed in a sufficient amount to produce 1,4-cyclohexanedimethanol, said 1,4-cyclohexanedimethanol pathway comprising an enzyme set selected from the group consisting of:
(1) a p-toluate monooxygenase, a p-hydroxymethyl benzoate reductase, a dihydroxymethyl benzene dehydrogenase;
(2) a p-toluate kinase, a (p-methylbenzoyloxy)phosphonate reductase (dephosphorylating), a p-methylbenzyl alcohol dehydrogenase, and a p-methylbenzyl alcohol monooxygenase;
(3) a p-methylbenzoyl-CoA synthetase, transferase and/or hydrolase, a phosphotrans-p-methylbenzoylase, a (p-methylbenzoyloxy)phosphoriate reductase (dephosphorylating), a p-methylbenzyl alcohol dehydrogenase, and a p-methylbenzyl alcohol monooxygenase;
(4) a p-methylbenzoyl-CoA synthetase, transferase and/or hydrolase, a p-methylbenzoyl-CoA reductase, a p-methylbenzyl alcohol dehydrogenase, and a p-methylbenzyl alcohol monooxygenase;
(5) a p-toluate reductase, a p-methylbenzyl alcohol dehydrogenase, and a p-methylbenzyl alcohol monooxygenase;
(6) a p-toluate monooxygenase, a p-hydroxymethyl benzoyl-CoA synthetase, transferase and/or hydrolase, a p-hydroxymethyl benzoyl-CoA reductase, a dihydroxymethyl benzene dehydrogenase, and chemical reduction;
(7) a p-toluate monooxygenase, a p-hydroxymethyl benzoate kinase, a (p-hydroxymethylbenzoyloxy)phosphonate reductase (dephosphorylating), a dihydroxymethyl benzene dehydrogenase, and chemical reduction;
(8) a p-toluate monooxygenase, a p-hydroxymethyl benzoate kinase, a phosphotrans-p-hydroxymethylbenzoylase, a p-hydroxymethyl benzoyl-CoA reductase, a dihydroxymethyl benzene dehydrogenase, and chemical reduction;
(9) a p-toluate monooxygenase, a p-hydroxymethyl benzoate kinase, a phosphotrans-p-hydroxymethylbenzoylase, a p-hydroxymethyl benzoyl-CoA reductase, a 4-hydroxymethyl cyclohex-1,5-diene carboxyl CoA reductase, a 4-hydroxymethyl cyclohex-1-ene carboxyl-CoA reductase, a 4-hydroxymethylcyclohexanoyl-CoA reductase, and a 4-hydroxymethyl cyclohexane carbaldehyde reductase; and
(10) a p-toluate monooxygenase, a p-hydroxymethyl benzoyl-CoA synthetase, transferase and/or hydrolase, a p-hydroxymethyl benzoyl-CoA reductase, a 4-hydroxymethyl cyclohex-1,5-diene carboxyl CoA reductase, a 4-hydroxymethyl cyclohex-1-ene carboxyl-CoA reductase, a 4-hydroxymethylcyclohexanoyl-CoA reductase, and a 4-hydroxymethyl cyclohexane carbaldehyde reductase.

2. The non-naturally occurring microbial organism of claim 1, wherein said microbial organism comprises two, three or four exogenous nucleic acids each encoding a 1,4-cyclohexanedimethanol pathway enzyme.

3. (canceled)

4. (canceled)

5. The non-naturally occurring microbial organism of claim 2, wherein said four exogenous nucleic acids encode enzyme set (1), (5), (6), or (7).

6. The non-naturally occurring microbial organism of claim 1, wherein said microbial organism comprises five exogenous nucleic acids each encoding a 1,4-cyclohexanedimethanol pathway enzyme.

7. The non-naturally occurring microbial organism of claim 6, wherein said five exogenous nucleic acids encode enzyme set (2), (4), or (8).

8. The non-naturally occurring microbial organism of claim 1, wherein said microbial organism comprises six exogenous nucleic acids each encoding a 1,4-cyclohexanedimethanol pathway enzyme.

9. The non-naturally occurring microbial organism of claim 8, wherein said six exogenous nucleic acids encode enzyme set (3).

10. The non-naturally occurring microbial organism of claim 1, wherein said microbial organism comprises seven exogenous nucleic acids each encoding a 1,4-cyclohexanedimethanol pathway enzyme.

11. The non-naturally occurring microbial organism of claim 10, wherein said seven exogenous nucleic acids encode enzyme set (9).

12. The non-naturally occurring microbial organism of claim 1, wherein said microbial organism comprises eight exogenous nucleic acids each encoding a 1,4-cyclohexanedimethanol pathway enzyme.

13. (canceled)

14. (canceled)

15. (canceled)

16. A method for producing 1,4-cyclohexanedimethanol, comprising culturing a non-naturally occurring microbial organism having a 1,4-cyclohexanedimethanol pathway, wherein said microbial organism comprises at least one exogenous nucleic acid encoding a 1,4-cyclohexanedimethanol pathway enzyme expressed in a sufficient amount to produce 1,4-cyclohexanedimethanol, under conditions and for a sufficient period of time to produce 1,4-cyclohexanedimethanol, said 1,4-cyclohexanedimethanol pathway comprising an enzyme set selected from the group consisting of:
(1) a p-toluate monooxygenase, and a p-hydroxymethyl benzoate reductase, a dihydroxymethyl benzene dehydrogenase;
(2) a p-toluate kinase, a (p-methylbenzoyloxy)phosphonate reductase (dephosphorylating), a p-methylbenzyl alcohol dehydrogenase, and a p-methylbenzyl alcohol monooxygenase;
(3) a p-methylbenzoyl-CoA synthetase, transferase and/or hydrolase, a phosphotrans-p-methylbenzoylase, a (p-methylbenzoyloxy)phosphonate reductase (dephosphorylating), a p-methylbenzyl alcohol dehydrogenase, and a p-methylbenzyl alcohol monooxygenase;
(4) a p-methylbenzoyl-CoA synthetase, transferase and/or hydrolase, a p-methylbenzoyl-CoA reductase, a p-methylbenzyl alcohol dehydrogenase, and a p-methylbenzyl alcohol monooxygenase;
(5) a p-toluate reductase, a p-methylbenzyl alcohol dehydrogenase, and a p-methylbenzyl alcohol monooxygenase;
(6) a p-toluate monooxygenase, a p-hydroxymethyl benzoyl-CoA synthetase, transferase and/or hydrolase, a p-hydroxymethyl benzoyl-CoA reductase, a dihydroxymethyl benzene dehydrogenase, and chemical reduction;
(7) a p-toluate monooxygenase, a p-hydroxymethyl benzoate kinase, a (p-hydroxymethylbenzoyloxy)phosphonate reductase (dephosphorylating), a dihydroxymethyl benzene dehdyrogenase, and chemical reduction;
(8) a p-toluate monooxygenase, a p-hydroxymethyl benzoate kinase, a phosphotrans-p-hydroxymethylbenzoylase, a p-hydroxymethyl benzoyl-CoA reductase, a dihydroxymethyl benzene dehdyrogenase, and chemical reduction;
(9) a p-toluate monooxygenase, a p-hydroxymethyl benzoate kinase, a phosphotrans-p-hydroxymethylbenzoylase, a p-hydroxymethyl benzoyl-CoA reductase, a 4-hydroxymethyl cyclohex-1,5-diene carboxyl CoA reductase, a 4-hydroxymethyl cyclohex-1-ene carboxyl-CoA reductase, a 4-hydroxymethylcyclohexanoyl-CoA reductase, and a 4-hydroxymethyl cyclohexane carbaldehyde reductase; and
(10) a p-toluate monooxygenase, a p-hydroxymethyl benzoyl-CoA synthetase, transferase and/or hydrolase, a p-hydroxymethyl benzoyl-CoA reductase, a 4-hydroxymethyl cyclohex-1,5-diene carboxyl CoA reductase, a 4-hydroxymethyl cyclohex-1-ene carboxyl-CoA reductase, a 4-hydroxymethylcyclohexanoyl-CoA reductase, and a 4-hydroxymethyl cyclohexane carbaldehyde reductase.

17. (canceled)

18. (canceled)

19. The method of claim 16, wherein said microbial organism comprises two, three or four exogenous nucleic acids each encoding a 1,4-cyclohexanedimethanol pathway enzyme.

20. (canceled)

21. (canceled)

22. The method of claim 19, wherein said four exogenous nucleic acids encode enzyme set (1), (5), (6), or (7).

23. The method of claim 16, wherein said microbial organism comprises five exogenous nucleic acids each encoding a 1,4-cyclohexanedimethanol pathway enzyme.

24. The method of claim 23, wherein said five exogenous nucleic acids encode enzyme set (2), (4), or (8).

25. The method of claim 16, wherein said microbial organism comprises six exogenous nucleic acids each encoding a 1,4-cyclohexanedimethanol pathway enzyme.

26. The method of claim 25, wherein said six exogenous nucleic acids encode enzyme set (3).

27. The method of claim 16, wherein said microbial organism comprises seven exogenous nucleic acids each encoding a 1,4-cyclohexanedimethanol pathway enzyme.

28. The method of claim 27, wherein said seven exogenous nucleic acids encode enzyme set (9).

29. The method of claim 16, wherein said microbial organism comprises eight exogenous nucleic acids each encoding a 1,4-cyclohexanedimethanol pathway enzyme.

30. (canceled)
